SEQUENCE LISTING (11 sequences)

SEQ ID NO: 1 - DNA, 42 bp, Artificial
  Prepared synthetically
  misc_feature (33)..(33): Biotinylated
cacgaatcaaagctctcaacgcctgcaagtcctaagacgcca

SEQ ID NO: 2 - DNA, 19 bp, Artificial
  Synthetic sequence
catggcaccaccagcatct

SEQ ID NO: 3 - DNA, 20 bp, Artificial
  Synthetic sequence
atccgcgtgtttcttttcga

SEQ ID NO: 4 - DNA, 25 bp, Artificial
  Synthetic sequence
  misc_feature (1)..(1): tris-dithiol phosphoramidite linker
aaaaaattgagagctttgattcgtg

SEQ ID NO: 5 - DNA, 42 bp, Artificial; identical to SEQ ID NO: 1
  Synthetic sequence
  misc_feature (1)..(1): tris-dithio phosphoramidite linkage
  misc_feature (42)..(42): Biotinylated

SEQ ID NO: 6 - DNA, 42 bp, Artificial; identical to SEQ ID NO: 1
  Synthetic sequence

SEQ ID NO: 7 - DNA, 45 bp, Artificial
  Synthetic sequence
aaaaaaaccccagcaattcaagtgtgttgagagctttgattcgtg

SEQ ID NO: 8 - DNA, 42 bp, Artificial; identical to SEQ ID NO: 1
  Synthetic sequence

SEQ ID NO: 9 - DNA, 25 bp, Artificial
  Synthetic sequence
  misc_feature (1)..(1): tris-dithio phosphoramidite hexa-ethylene glycol linkage
ttgagagctttgattcgtgaaaaaa

SEQ ID NO: 10 - DNA, 37 bp, Artificial
  Synthetic sequence
atcaaagctctcaacgcctgcaagtcctaagacgcca

SEQ ID NO: 11 - DNA, 25 bp, Artificial
  Synthetic sequence
  misc_feature (1)..(1): tris-dithio phosphoramidite hexa-ethylene glycol linkage
ttgagagctttgattcgtgaaaaaa

(19) United States
(12) Patent Application Publication
(10) Pub. No.: US 2007/0099211 A1
(43) Pub. Date: May 3, 2007

(54) Detection of nucleic acid amplification

(76) Inventors: Vissarion Aivazachvili, Oakland, CA (US); Kristian M. Scaboo, Castro Valley, CA (US); Aldrich N.K. Lau, Palo Alto, CA (US); Konrad Faulstich, Salem-Neufrach (DE); Robert G. Eason, Los Gatos, CA (US); John R. Van Camp, San Ramon, CA (US); Timothy Z. Liu, Fremont, CA (US)

Correspondence Address: KOLISCH HARTWELL, P.C., 200 PACIFIC BUILDING, 520 SW YAMHILL STREET, PORTLAND, OR, 97204, US

(21) Appl. No.: 11/488,439

(22) Filed: Jul. 17, 2006

Related U.S. Application Data
(60) Provisional application No. 60/699,950, filed on Jul. 15, 2005.
(60) Provisional application No. 60/749,003, filed on Dec. 9, 2005.

(51) Int. Cl. C12Q 1/68; C12P 19/34

(52) U.S. Cl. 435/6; 435/91.2

(57) ABSTRACT

Methods for detecting a target polynucleotide sequences are provided that utilize a probe having a target-complementary segment and a detectable tag. By cleaving the detectable tab and associating the tag with a tag complement coupled to an electrode, an electrochemical signal can be detected that is related to the presence of the tag:tag complement complex.

This application is based upon and claims benefit under 35 U.S.C. § 119 of U.S. Provisional Patent Application Ser. No. 60/699,950, filed Jul. 15, 2005; and U.S. Provisional Patent Application Ser. No. 60/749,003, filed Dec. 9, 2005; each which is hereby incorporated by reference.

### INTRODUCTION

Nucleic acid detection may be performed by a variety of assay formats. Such assays may be qualitative, for example when used to evaluate a biological sample. However, a wide variety of biological applications could be improved by the ability to detect target nucleic acids without requiring either cumbersome blotting techniques, or the expensive and delicate equipment typically required for optical methods.

### BRIEF DESCRIPTION OF THE DRAWINGS

FIG. 1 is a scheme depicting an exemplary amplification probe hybridized to a polynucleotide sequence that is an amplicon template, according to an embodiment of the invention.

FIG. 2 is a scheme depicting cleavage of a flap moiety from a hybridized nucleic acid complex.

FIG. 3 is a scheme depicting the monitoring of the polymerase chain reaction (PCR) using a detection oligonucleotide, according to an embodiment of the invention.

FIG. 4 is a scheme depicting the use of a nucleic acid sequence that is capable of self-priming its own extension during amplification, according to an embodiment of the invention.

FIG. 5 is a scheme depicting the use of a nucleic acid sequence that includes a hairpin loop that contains a tag sequence, according to an embodiment of the invention.

FIG. 6 is a scheme depicting the use of a cleavable tag sequence complementary to a detection oligonucleotide during amplification, according to an embodiment of the invention.

FIG. 7 is a schematic depiction showing detection of a tag sequence remote from the cleavage location, by virtue of interactions between the cleaved tag and an electrode surface.

FIG. 8 is a schematic depiction of a microfluidic system, according to an embodiment of the invention.

FIG. 9 shows that the presence of a tag sequence does not influence PCR amplification of a target sequence, as verified by electrophoretic analysis of the amplicon compared to control reactions, as described in Example 1.

FIG. 10 shows the electrochemical detection of a tag sequence, as described in Example 1.

FIG. 11 shows another example of electrochemical detection of a tag sequence, as described in Example 2.

FIG. 12 is a schematic showing tag detection at an electrode via electrostatically bound redox centers, as discussed in Example 4.

FIG. 13 is a voltammogram showing the electrochemical response of a mediator compound, as described in Example 7.

FIG. 14 is a plot of integrated charge vs. DNA concentration for compounds 1 and 7, as described in Example 7.

FIG. 15 shows cyclic voltammograms of an amplification probe and of Compound 21, as described in Example 10.

FIG. 16 shows cyclic voltammograms of an amplification probe and of Compound 7, as described in Example 10.

FIG. 17 shows the differentiation between detection of cleaved and uncleaved tag sequences for tag sequences 1, 2, and 3, as described in Example 11.

### DESCRIPTION OF VARIOUS EMBODIMENTS

The present description is directed methods of systems for detecting target polynucleotide sequences. The method can include contacting a probe with a sample comprising at least one target polynucleotide sequence, under conditions effective for the probe to form a probe-target complex, where the probe itself comprises a target-complementary segment and a detectable tag. The detectable tag can then be cleaved from the probe, with the released tag then associating with a tag complement that is coupled to an electrode. The electrochemical signal that is detected due to the immobilized tag:tag complement complex immobilized at the electrode can be correlated with the presence of the target polynucleotide sequence in the sample.

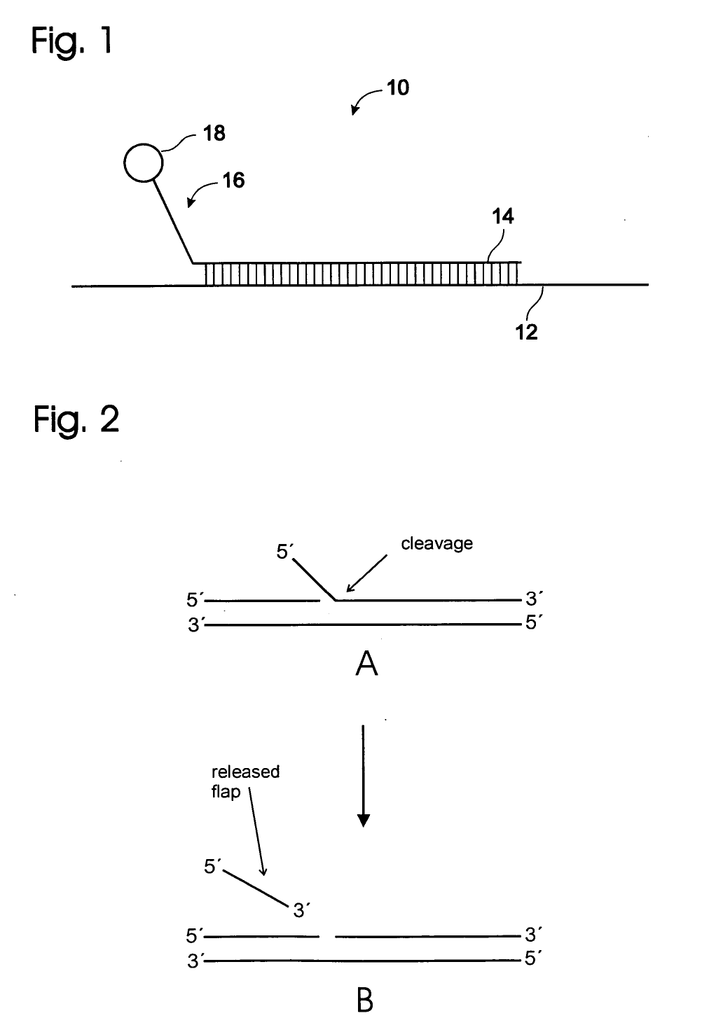

In some embodiments, the present description includes a method for detection of the amplification of a nucleic acid. In the method, with reference to FIG. 1, an amplification probe 10 is hybridized to a polynucleotide sequence 12 that is an amplicon template, or target, for a polynucleotide amplification process. The amplification probe 10 includes a complementary polynucleotide sequence 14 and one or more detection tags 16. During the amplification process, enzyme action cleaves one or more detection tags from the complementary sequence. Detection of the cleaved detection tag in turn detects the cleavage event, and therefore the replication of the amplicon template, as shown in FIG. 1.

‘Hybridization’, as used herein, refers to the association of two polynucleotide sequences to form a stable double-stranded structure through hydrogen bonding between bases in the two sequences. A sequence may be considered ‘complementary’ to a second sequence even though the two sequences are not completely and exactly complementary, provided that the sequences include regions of sufficient complementarity that the resulting hybrid is stable under standard laboratory conditions. Any complementary sequence that is capable of at least substantially selective hybridization to the amplicon template is a suitable complementary sequence for the purposes of the method. Typically the complementary sequence is composed of nucleotides and/or analogs thereof, and has sufficient length to confer at least some binding specificity to the amplification probe. The complementary sequence can include RNA or DNA, or a mixture or a hybrid thereof. The complementary sequence can include a natural nucleic acid polymer (biological in origin) or a synthetic nucleic acid polymer (modified or prepared artificially).

The complementary sequence can have any suitable natural and/or artificial structure. The nucleic acid can include a phosphodiester backbone such that the nucleic acid has a negative charge in aqueous solutions of neutral pH. A phosphodiester backbone generally includes a sugar-phosphate backbone of alternating sugar and phosphate moieties, with a nucleotide base (generally, a purine or a pyrimidine group) attached to each sugar moiety. Any sugar(s) can be included in the backbone including ribose (for RNA), deoxyribose (for DNA), arabinose, hexose, 2′-fluororibose, and/or a structural analog of a sugar, among others. The nucleic acid analytes and/or probes of the present teachings can be analogs including any suitable alternative backbone. Exemplary alternative backbones can be less negatively charged than a phosphodiester backbone and can be substantially uncharged (neither positively nor negatively charged). Exemplary alternative backbones can include phosphoramides, phosphorothioates, phosphorodithioates, O-methylphosphoroamidites, peptide nucleic acids (including N-(2-aminoethyl)glycine backbone units), locked nucleic acids (e.g., see Koshkin et al., Tetrahedron 54:3607-30 (1998), WO 98/39352, WO 99/14226, WO 00/56746, and WO 99/60855, each hereby incorporated by reference), positively charged backbones, non-ribose backbones, etc. Nucleic acids with artificial backbones and/or moieties can be suitable, for example, to increase or reduce the total charge, increase or reduce base-pairing stability, increase or reduce chemical stability, to alter the ability to be acted on by a reagent, and/or the like. In exemplary embodiments, nucleic acid probes (such as peptide nucleic acids) with a reduced negative charge can be employed with phosphodiester-based analytes to increase the sensitivity of optical elements for detection of the analytes.

The complementary sequence optionally contains one or more modified bases or links or contains labels that are non-covalently or covalently attached. For example, the modified base can be a naturally occurring modified base or a synthetically altered base. Where the nucleic acid includes modified nucleotide bases, the bases can include, without limitation, adenine, cytosine, guanine, thymine, uracil, inosine, 2-amino adenine, 2-thiothymine, 3-methyl adenine, C5-bromouracil, C5-fluorouracil, C5-iodouracil, C5-methyl cytosine, 7-deazaadenine, 7-deazaguanine, 8-oxoadenine, 8-oxoguanine, 2-thiocytosine, 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, beta-D-galactosylqueuosine, 2′-O-methylguanosine, N6-isopentenyladenosine, 1-methyl-adenosine, 1-methylpseudouridine, 1-methylguanosine, 1-methylinosine, 2,2-dimethyl-guanosine, 2-methyladenosine, 2-methylguanosine, 3-methylcytidine, 5-methylcytidine, N6-methyladenosine, 7-methylguanosine, 5-methylaminomethyluridine, 5-methoxy-aminomethyl-2-thiouridine, beta-D-mannosylqueuosine, 5-methoxycarbonylmethyl-2-thiouridine, 5-methoxycarbonylmethyluridine, 5-methoxyuridine 2-methylthio-N6-isopentenyladenosine, N-((9-beta-D-ribofuranosyl-2-methylthiopurine-6-yl)carbamoyl)-threonine, N-((9-beta-D-ribofuranosylpurine-6-yl)N-methylcarbamoyl)threonine, uridine-5-oxyacetic acid-methylester, uridine-5-oxyacetic acid, wybutoxosine, pseudouridine, queuosine, 5-methyl-2-thiouridine, 2-thiocytidine, 5-methyl-2-thiouridine, 2-thiouridine 4-thiouridine, 5-methyluridine N-((9-beta-D-ribofuranosylpurine-6-yl)-carbamoyl)-threonine, 2′-O-methyl-5-methyluridine, 2′-O-methyluridine, wybutosine, 3-(3-amino-3-carboxy-propyl)uridine, and (acp3)u.

In addition to the presence of one or more detection tags, the probe can include a reactive functional group, or be substituted by a conjugated substance, for example in order to facilitate partial or complete removal of the uncleaved probe from a mixture of components. In particular, the probe can be modified in order to facilitate separation of uncleaved probe from the cleaved detection tag. For example, the complementary probe can be modified at the 3′-terminus with biotin, so that the cleaved complementary probe can be immobilized by a streptavidin-modified surface or substrate.

By detecting the cleaved detection tag, the replication of the target amplicon can be detected. Probe cleavage may be performed using any method that is compatible with the purposes of the invention. Exemplary, non-limiting examples for performing probe cleavage include 5′-nuclease methodologies (e.g., Gelfand et al., U.S. Pat. Nos. 5,210,015 and 5,487,972), such as detailed further herein, INVADER™ methodologies, (e.g., Prudent et al., U.S. Pat. Nos. 5,985,557, 5,994,069, and 6,090,543), and FEN-LCR methodologies (e.g., Bi et al., U.S. Pat. No. 6,511,810). In the INVADER type formats, a pair of oligonucleotides are provided that bind to adjacent sequences in a target polynucleotide to form a cleavage complex wherein the 3′ end of the target-complementary portion of a first oligonucleotide is immediately adjacent to or overlaps with the 5′ end of the target-complementary portion of a second oligonucleotide. The complex is recognized by enzymes that contain flap endonuclease activity, also known as 5′ nuclease activity, which cleave the second oligonucleotide on the 5′ side of the 5′-most complementary nucleotide that is adjacent to the 3′-most nucleotide of the target-complementary segment of the first probe. In embodiments in which the first probe contains one or more non-complementary nucleotide attached to the 3′ end of its target-complementary sequence, cleavage of the second probe occurs on the 3′ side of the 5′-most complementary nucleotide. In some embodiments, cleaved second probe can be replaced by a new uncleaved probe to generate additional cleaved probe. In FEN-LCR methods, first and second oligonucleotides can be ligated together after the second oligonucleotide has been cleaved, to produce new copies of amplicon for linear or exponential amplification. Other methods that may be useful in the present invention include probe cleavage methods such as disclosed in Walder (U.S. Pat. No. 5,403,711) and Duck (U.S. Pat. No. 5,011,769), for example, in which RNA-containing probes are cleaved with RNAse H, or wherein probes that contain an abasic subunit can be cleaved by an appropriate endonuclease such as endonuclease IV from E. coli, for example. Another example of methods for probe cleavage is recombinase polymerase amplification (RPA) such as described in Piepenburg et al. (PLoS Biology 4:1115-1121 (2006)) and coworkers (e.g., US Patent Pub. 2005/0112631 and PCT Pub. WO 03/072805), when modified to include binding of a cleavable probe to the amplified target. Further cleavage methods and enzymes are also disclosed in U.S. Pat. Nos. 5,869,245 (Yeung) and 5,698,400 (Cotton et al.). In all cases, probe cleavage produces a detectable tag that comprises one or more electrochemical moieties, one or more binding partners for subsequent detection, or that is detectable using an electrochemical moiety that interacts with the detectable tag, such as illustrated herein.

The detection tag can include one or more detectable labels 18. By detectable label is meant any moiety that can be detected and/or quantitated. The detection tag can be detected either directly or indirectly. Where the detection tag is detected directly, the detection tag optionally includes a detectable label such as an electrochemical moiety.

Alternatively, the detection tag is detected indirectly, for example by the interaction of the detection tag with an additional detection reagent. For example, the detection tag may include a member of a specific binding pair, such as a hapten for a labeled antibody, or a nucleic acid sequence that is labeled by a complementary sequence. The detection tag may include a digoxigenin moiety, for example, that can be used as a target for an antibody labeled with an electrochemical moiety. The additional detection reagent can include an electrochemical moiety, so that association of the reagent with the detection tag facilitates electrochemical detection of the detection tag.

In some embodiments, the invention comprises amplification of a target via electrochemical detection, optionally in the presence of an electrochemical moiety. The electrochemical moiety can be bound as a label on the detection tag, or it may be present as a detection reagent that interacts with the detection tag. The electrochemical moiety may be any moiety that can transfer electrons to or from an electrode. The selection of moiety will be dependent upon the particular composition of the probe chosen. Particularly preferred moieties include transition metal complexes. Suitable transition metal complexes include, for example, ruthenium²⁺ (2,2′-bipyridine)₃ (Ru(bpy)₃²⁺), ruthenium²⁺(4,4′-dimethyl-2,2′-bipyridine)₃ (Ru(Me²-bpy)₃²⁺), ruthenium²⁺(5,6-dimethyl-1,10-phenanthroline)₃ (Ru(Me₂-phen)₃²⁺), iron²⁺(2,2′-bipyridine)₃ (Fe(bpy)₃²⁺), iron²⁺(5-chlorophenanthroline)₃ (Fe(5-Cl-phen)₃²⁺), osmium²⁺(5-chlorophenanthroline)₃ (Os(5-Cl-phen)₃²⁺), osmium²⁺(2,2′-bipyridine)₂ (imidazolyl), dioxorhenium¹⁺ phosphine, and dioxorhenium¹⁺ pyridine (ReO₂(py)₄¹⁺). Some anionic complexes useful as moieties are: Ru(bpy)((SO₃)₂-bpy)₂²⁻ and Ru(bpy)((CO₂)₂-bpy)₂²⁻ and some zwitterionic complexes useful as moieties are Ru(bpy)₂ ((SO₃)₂-bpy) and Ru(bpy)₂((CO₂)₂-bpy) where (SO₃)₂-bpy₂- is 4,4′-disulfonato-2,2′-bipyridine and (CO₂)₂-bpy₂- is 4,4′-dicarboxy-2,2′-bipyridine. Suitable substituted derivatives of the pyridine, bypyridine and phenanthroline groups may also be employed in complexes with any of the foregoing metals. Suitable substituted derivatives include but are not limited to 4-aminopyridine, 4-dimethylpyridine, 4-acetylpyridine, 4-nitropyridine, 4,4′-diamino-2,2′-bipyridine, 5,5′-diamino-2,2′-bipyridine, 6,6′-diamino-2,2′-bipyridine, 4,4′-diethylenediamine-2,2′-bipyridine, 5,5′-diethylenediamine-2,2′-bipyridine, 6,6′-diethylenediamine-2,2′-bipyridine, 4,4′-dihydroxyl-2,2′-bipyridine, 5,5′-dihydroxyl-2,2′-bipyridine, 6,6′-dihydroxyl-2,2′-bipyridine, 4,4′, 4″-triamino-2,2′,2″-terpyridine, 4,4′,4″-triethylenediamine-2,2′,2″-terpyridine, 4,4′,4″-trihydroxy-2,2′,2′-terpyridine, 4,4′,4″-trinitro-2,2′,2″-terpyridine, 4,4′,4″-triphenyl-2,2′,2″-terpyridine, 4,7-diamino-1,10-phenanthroline, 3,8-diamino-1,10-phenanthroline, 4,7-diethylenediamine-1,10-phenanthroline, 3,8-diethylenediamine-1,10-phenanthroline, 4,7-dihydroxyl-1,10-phenanthroline, 3,8-dihydroxyl-1,10-phenanthroline, 4,7-dinitro-1,10-phenanthroline, 3,8-dinitro-1,10-phenanthroline, 4,7-diphenyl-1,10-phenanthroline, 3,8-diphenyl-1,10-phenanthroline, 4,7-disperamine-1,10-phenanthroline, 3,8-disperamine-1,10-phenanthroline, dipyrido[3,2-a:2′,2′-c]phenazine, and 6,6′-dichloro-2,2′-bipyridine, among others.

In order to facilitate detection, the detection tag resulting from probe cleavage may be separated from uncleaved probe. The separation step can be by simple diffusion, where either the detection tag or the complementary sequence is tethered or bound in place, such that the cleaved products can diffuse away. Alternatively, or in addition, either one or more of the cleaved products, or the uncleaved probe can be mechanically separated from the reaction mixture. In one aspect, the complementary sequence includes a functional group, such as biotin, that facilitates removal of the complementary sequence, and therefore uncleaved probe, from the reaction mixture. Where the complementary sequence is functionalized with a biotin moiety, mixing the reaction mixture with streptavidin-coated beads, or passing the reaction mixture through a streptavidin-modified matrix, for example, serves to capture the complementary sequence and uncleaved probe and facilitates detection of cleaved detection tag.

In some embodiments, the detection tag comprises a tag sequence. The tag sequence may comprise a polynucleotide, and can include any nucleic acid composition recited above for the complementary sequence. In some embodiments, the tag sequence is selected so that it does not hybridize or otherwise associate with an amplicon template. Additionally, the tag sequence typically is joined to the complementary probe with a connection that is cleaved by enzyme action. Preferably the tag sequence can be cleaved by an enzyme that facilitates nucleic acid amplification, in particular amplification of the amplicon template, such as PCR. For example, the tag sequence is readily cleaved from the probe by 5′ nuclease activity of a DNA polymerase. In some embodiments, both the tag sequence and the complementary sequence are DNA sequences. For example, the tag sequence can include about 14 to about 40 bases. Alternatively, the tag sequence can include about 14 to about 20 bases. In a particular aspect of the method, the tag sequence is 19 bases long.

In some embodiments, such tag sequences may be produced using an enzyme comprising a 5′-endonuclease or 5′-exonuclease activity, such as a flap endonuclease or a DNA polymerase having such activity, to remove a flap moiety from an appropriate hybridization complex. For example, such a complex may have the form illustrated at A in the scheme shown in FIG. 2.

The complex (“cleavage complex”) designated A in FIG. 2 includes a polynucleotide strand (the “target strand”) that is or contains a target sequence (“amplicon template”), illustrated here in the 3′ to 5′ direction left-to-right. Hybridized to the 3′ side of the amplicon template is an upstream polynucleotide having a 5′ end on the left of complex A and a 3′ end (not marked). In some embodiments, an upstream polynucleotide can be produced in situ by primer extension during a polymerase chain reaction (PCR). In other embodiments, the upstream polynucleotide can be provided as an intact species without further modification or extension. Hybridized on the right (5′) side of the amplicon template is a cleavable probe that contains a template-binding segment that is hybridized to a complementary sequence in the amplicon template, and a tag sequence (or simply, “tag”) that is not hybridized to the amplicon template and that is linked to the 5′ end of the template-binding segment of the cleavable probe. Reaction of the cleavage complex with a suitable enzyme such as noted above provides a cleaved complex (designated B in FIG. 2) comprising the amplicon template, an upstream polynucleotide, and the template-binding segment from the cleavable probe. Also produced is a tag sequence that can be released from the complex for subsequent detection as discussed further herein.

In some embodiments, a tag sequence can be generated in a 5′ nuclease polymerase chain reaction. A reaction mixture is provided that comprises first and second primers that are complementary to opposite ends of a duplex target sequence to be amplified, such that the first primer can initiate, by polymerase-mediated primer extension, synthesis of a strand that is complementary to the amplicon template strand to which the first primer hybridizes, and the second primer can initiate, by polymerase-mediated primer extension, polymerase-mediated synthesis of the amplicon template strand or a copy thereof. The reaction mixture also comprises a cleavage probe, such as described above, having an amplicon template-binding segment that binds to a complementary sequence in the amplicon template that is located between the sequences to which the first and second primers bind. When nucleotide triphosphates are present, the cleavage probe is preferably rendered non-extendable at its 3′ end by, for example, substitution of the 3′ hydroxyl in the ribose or deoxyribose ring of the 3′ terminal nucleotide subunit with hydrogen, fluorine, amino, or other non-hydroxyl moiety, or by blockage of the 3′ hydroxyl group with a blocking group such as 3′ amino, 3′ fluoro, 3′ H, 3′-phosphoate, 3′ methyl, 3′-tert-butyl, or 3′ trityl.

For example, when the first primer and the cleavage probe both hybridize to the amplicon template strand, the primer can be extended in the presence of nucleotide triphosphates (NTPs) such as ATP, CTP, GTP, and TTP, or analogs thereof, using a template-dependent DNA polymerase having 5′ nuclease activity. When the primer has been extended such that its 3′ end is adjacent to, or overlaps with, the 5′ end of the cleavage probe (see Scheme I at A above), the probe is cleaved by the nuclease activity of the polymerase, thereby releasing the flap moiety from the cleavage complex (see Scheme II at B). As the primer is extended through and past the cleaved cleavage probe, the polymerase may cleave the template-binding segment of the probe at additional sites until the remainder of the template-binding segment dissociates from the amplicon template. The extended primer may then serve as a template for the second primer to replicate the original (first) amplicon template strand.

The primers and probes used in the PCR example above may have any of a variety of lengths and configurations suitable for producing a detectable flap to be detected by methods herein. Typically, primers may be from about 18 to about 30 nucleotides in length, or from 20 to 25 nucleotides, although lengths outside of these ranges may also be used. For example, shorter lengths can be used when a primer contains one or more nucleotide analogs having enhanced basepairing affinities relative to DNA or RNA are used, such as locked nucleic acids (LNAs) or peptide nucleic acids (PNAs). The template-binding segment of a probe may likewise be of any suitable length, typically between 8 and 30 nucleotides, for example. When the probe also contains a polynucleotide flap moiety, the flap moiety may comprise a polynucleotide sequence of any suitable length, such as 10 to 40 nucleotides, depending on the desired specificity and sensitivity of detection.

The first and second primers may be designed to bind to produce an amplified product of any desired length, usually at least 30 or at least 50 nucleotides in length and up to 200, 300, 500, 1000, or more nucleotides in length. The probes and primers may be provided at any suitable concentrations. For example, forward and reverse primers may be provided at concentrations typically less than or equal to 500 nM, such as from 20 nM to 500 nm, or 50 to 500 nM, or from 100 to 500 nM, or from 50 to 200 nM.

In some embodiments, probes are typically provided at concentrations less than or equal to 1000 nM, such as from 20 nM to 500 nm, or 50 to 500 nM, or from 100 to 500 nM, or from 50 to 200 nM. Exemplary conditions for concentrations of NTPs, enzyme, primers and probes can also be found in U.S. Pat. No. 5,538,848 (hereby incorporated by reference), or can be achieved using commercially available reaction components (e.g., as can be obtained from Applied Biosystems, Foster City, Calif.).

In one aspect of the method, the tag sequence is modified by being associated with a detectable label. The tag sequence can be labeled at the 5′-terminus with an electrochemically active moiety, or a member of a specific binding pair. Alternatively, the tag sequence can bind with or otherwise become associated with a detection reagent after being cleaved. As discussed above, the cleaved tag sequence can be detected either in solution, or after capture or immobilization. Optionally, the method includes a separation step to prevent uncleaved tag sequences from interfering with detection of cleaved tag sequences. The foregoing teachings also apply to tags that do not comprise polynucleotide sequences.

In one aspect of the method, after enzymatic cleavage, the tag sequence is captured and/or immobilized. Where the tag sequence includes a detectable label, the detectable label is then detected. Where the tag sequence is subsequently labeled with a detection reagent, the detection reagent can label or complex with the immobilized tag sequence.

The tag sequence can be immobilized as a result of either specific or non-specific interactions. For example, the tag sequence can be derivatized with a member of a specific binding pair which specifically binds to and is complementary with a particular spatial and polar organization of the other member of that specific binding pair. Representative specific binding pairs may include ligands and receptors, and may include but are not limited to the following pairs: antigen-antibody, biotin-avidin, biotin-streptavidin, IgG-protein A, IgG-protein G, carbohydrate-lectin, enzyme-enzyme substrate; DNA-antisense DNA, and RNA-antisense RNA.

Where the tag sequence is or includes a nucleic acid sequence, the tag sequence itself may be captured and/or immobilized by a tag complement sequence that is substantially complementary with the tag sequence. For example, the cleaved tag sequence can be captured and immobilized by a capture antisense oligonucleotide that is itself immobilized on a surface or other substrate.

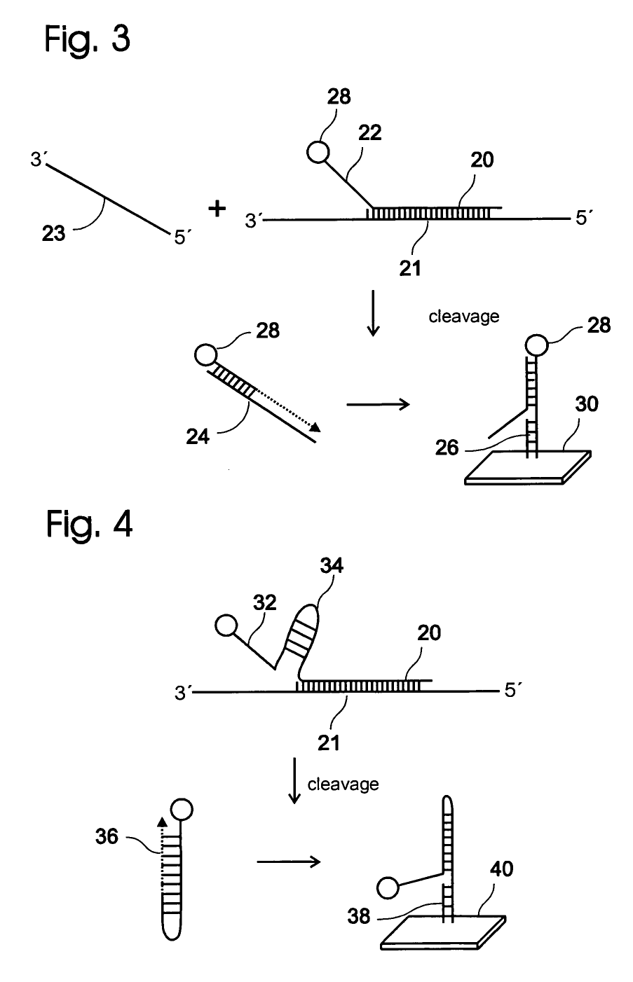

The use of antisense oligo sequences as capture sequences (tag complements) can allow the method to be multiplexed, for example by designing a plurality of complementary probes, each having a characteristic tag sequence. An array of capture oligonucleotides that are individually and respectively complementary to the selected tag sequences may be used to localize and capture individual tag sequences in a plurality of discrete detection zones.

Where the complementary sequence 20 that is complementary to the target sequence 21 includes a complementary polynucleotide sequence 22, the tag sequence can be formulated to include an additional sequence that is complementary to that of an additional detection oligonucleotide 23 as shown in FIG. 3. By permitting the PCR reaction mixture to cool during the PCR cycle, the additional detection oligonucleotide can hybridize with the cleaved tag sequence, as well as tag sequence that may still be bound to the complementary probe. The newly formed duplex 24 of the cleaved tag sequence and the additional detection oligonucleotide will extend at the free 3′ end, resulting in the formation of more stable ds-DNA, while the duplex with the non-cleaved tag sequence remains non productive and just dissociates after a subsequent increase in temperature.

The extension of the tag sequence generates a new sequence that is complementary to a sequence immobilized at a remote detection location. In one aspect, the immobilized sequence 26 is a peptide nucleic acid (PNA) sequence, so that it can not interfere with the PCR process. Binding of the extended tag sequence can be detected as discussed above, either by the presence of a detectable label 28 on the tag sequence itself, typically at either the 3′ or the 5′ terminus, or by subsequent association of a detectable label with the tag sequence, or the tag sequence in association with the immobilized sequence. In addition, FIG. 3 can also be used in non-PCR embodiments.

In one aspect, the immobilized sequence is a PNA sequence that is immobilized on a gold electrode 30, and the presence of the cleaved and extended tag sequence is detected by virtue of a detectable electroactive label 28 at the 5′ terminus of the tag sequence.

In another aspect, the tag sequence 32 includes a sequence of inverted repeats selected to form a stable loop-stem structure 34, as shown in FIG. 4. Therefore, upon cleaving the tag sequence from the complementary probe, the 3′-end of the tag sequence is capable of self-priming its own extension 36. The self-priming process can generate a new sequence complementary to an immobilized sequence 38, which may be a PNA sequence, and may be immobilized on an electrode 40.

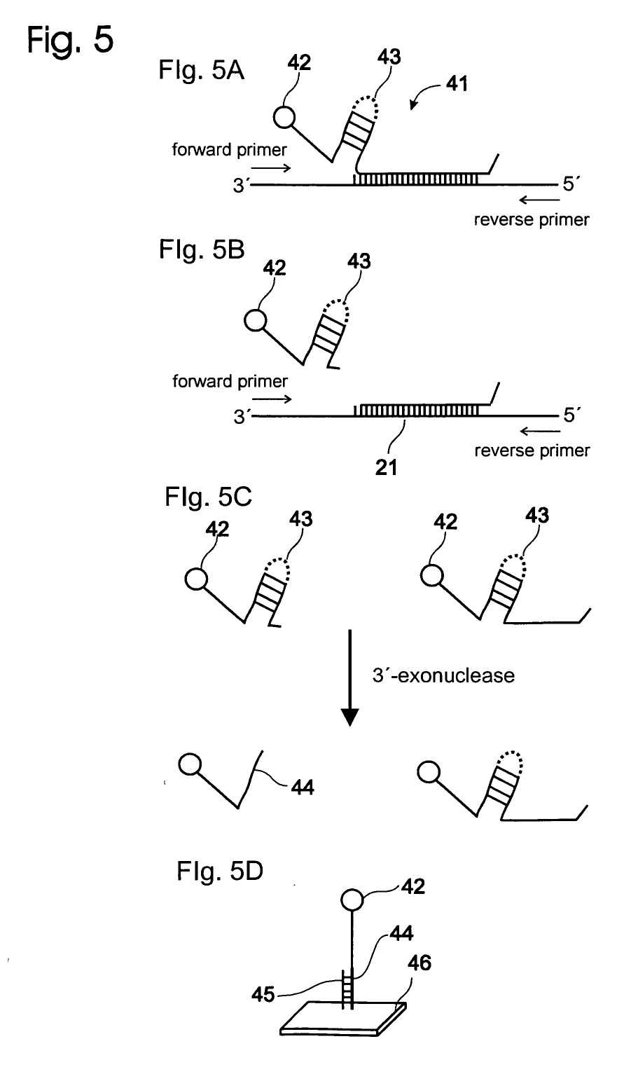

In another aspect, the present disclosure provides methods that utilize probes containing hairpin loops that include zipcode sequences. A schematic illustration is shown in FIGS. 5A-5C. The probe 41 is labeled with an electroactive label 42 and includes two polynucleotide sequences linked together by a spacer 43. The hydroxyl group at the 3′ end of the probe is typically protected from 3′ exonuclease digestion by a 3′ blocking group such as an acetyl or phosphate group, among others (as shown in FIG. 5A). The probe can additionally include a tag sequence that is cleaved by 5′ nuclease activity, e.g., the 5′ nuclease activity of a DNA polymerase during PCR, resulting in an unbound tag sequence (as shown in FIG. 5B). The cleaved flap has a 3′-OH group. Subsequently the 3′ end of the cleaved flap can be digested by a 3′ exonuclease, such as Exonuclease III (as shown in FIG. 5C). Exo III has a double-strand specific 3 ′-5′ exo-deoxyribonuclease activity but will also act on 3′ overhangs having fewer than 4 bases. Exo III can be deactivated by heating at 80° C. after a digestion step, if desired. The 3′ exonuclease digestion stops at spacer 43, which can simply be an organic linker, for example. At the other side of the spacer is a tag sequence 44 unique to that probe. Thus, after the tag sequence on the cleaved flap becomes single stranded, it can hybridize to a complementary immobilized sequence 45, for example bound to an electrode surface 46. (as shown in FIG. 5D). The tag sequence in uncleaved probes are not available for hybridization due to the hairpin loop. Therefore, there is no need to separate the cleaved and uncleaved probes prior to detection. The 3′ exonuclease can be added to a PCR reaction after thermal cycling, or can be deposited in a detection chamber that comprises the electrode(s). Captured flaps can be electrochemically detected on the electrode as described herein.

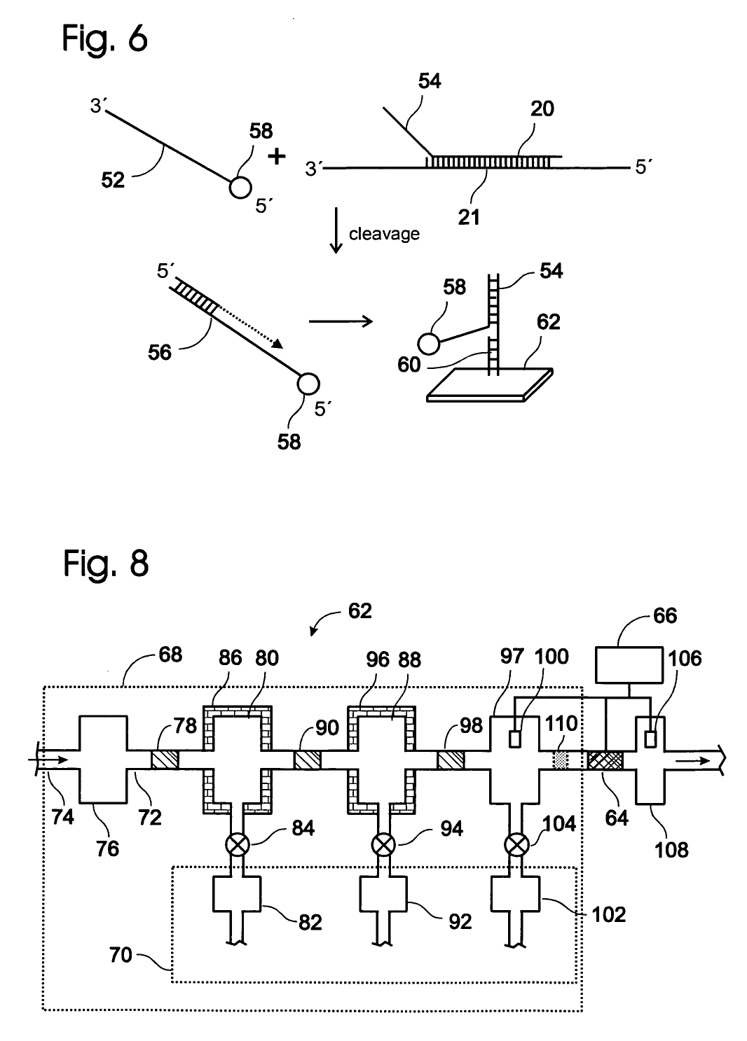

In another aspect, a detection oligo 52 complementary to the tag sequence 54 but longer is added, and upon cleavage of the tag sequence the generated 3′-OH group can be extended complementary to the detection oligonucleotide after annealing of the cleaved tag sequence with the detection oligonucleotide as shown by hybridized complex 56 in FIG. 6. A detectable label can either be attached to the tag sequence (as shown at 28 in FIG. 3) or a detectable label can be attached to the detection oligonucleotide (as shown at 58 in FIG. 6). As discussed previously, the new sequence may be complementary to, and therefore bind to, an immobilized sequence 60, which may be a PNA sequence, and which may be immobilized on an electrode 62.

The tag:tag complement complex may be detected by any of a variety of suitable mechanisms. In general, the tag is selected to bind or form a complex with the tag complement by specific covalent or non-covalent interactions (e.g., by hydrogen-bonding, ion-pairing or van der Waals attraction) under the conditions of detection, as opposed to merely passive interaction or diffusion into or through a size-exclusive porous matrix or barrier. Such specific interactions between the tag and tag complement can provide an additional level of specificity to increase signal to noise. Additionally, the detection conditions may also optionally include an electrochemical mediator or substrate by which the signal from an electrochemical moiety can be amplified. Where an electrochemically active moiety is present on the tag or tag complement, such a moiety may function as a mediator. Alternatively, one or more electrochemical mediators may be present in solution. An example of such a mediator is ascorbic acid. However, such mediators are not required for operation of the invention when the electrochemical moiety itself provides adequate signal.

In some embodiments, the probe is covalently attached to a surface or substrate in contact with a solution in which probe cleavage is occurring. In PCR embodiments, the denatured amplicon is able to hybridize to the complementary probe. Appropriate primers can then anneal to the amplicon, and extension of the amplicon sequence can proceed. During the extension, enzyme activity can result in cleavage of the complementary probe. For example, where amplification is carried out by PCR, the complementary probe is cleaved by the endonuclease activity of the polymerase enzyme.

Where the complementary probe is labeled with one or more tag sequences, the cleavage of the complementary probe results in liberation of the tag sequences. The extent of the reaction can then be determined by the presence or quantification of tag sequence in the reaction solution. Where the tag sequence is or comprises an electrochemically active label, the progress of the reaction can be determined electrochemically.

Where the tag sequence is detected at a remote location, it can be helpful for either the tag sequence, the remote location, or both, to be modified in such a way to increase interaction between the remote detection site and the tag sequence. Where the tag sequence comprises an electrochemically active label, the remote detection site can be an electrode surface. Alternatively, the tag sequence can be localized to the remote surface, and subsequently associated with an electrochemically active tag. For example, where the tag sequence includes a polynucleotide, the electrochemically active tag can selectively bind to polynucleotides, such as where the electrochemically active moiety is incorporated in an intercalating agent that binds to polynucleotides (as discussed in Example 2).

Where the tag sequence includes an electrochemically active label, the label can be detected directly, or it can be detected via one or more intermediate oxidation-reduction (redox) active species. Such a redox active species may shuttle electrons from an electrode surface to an electrochemically active label, or may shuttle electrons to yet another redox active shuttle species.

The tag sequence can be modified by inclusion of one or more capture moieties, in order to enhance interaction with the remote location. In one aspect, the remote location includes a gold metal surface, and the capture moieties include thiol or disulfide functional groups. The affinity between the sulfur-containing functional groups and the gold surface result in binding of the tag sequence. The tag sequence can include a polymeric or dendritic structure, including multiple thiol-containing functional groups, in order to maximize binding to the gold surface. The electrochemically active group can be incorporated into the thiolated tag sequence, or can be associated with the tag sequence before or after adsorption to the gold surface (see Example 7).

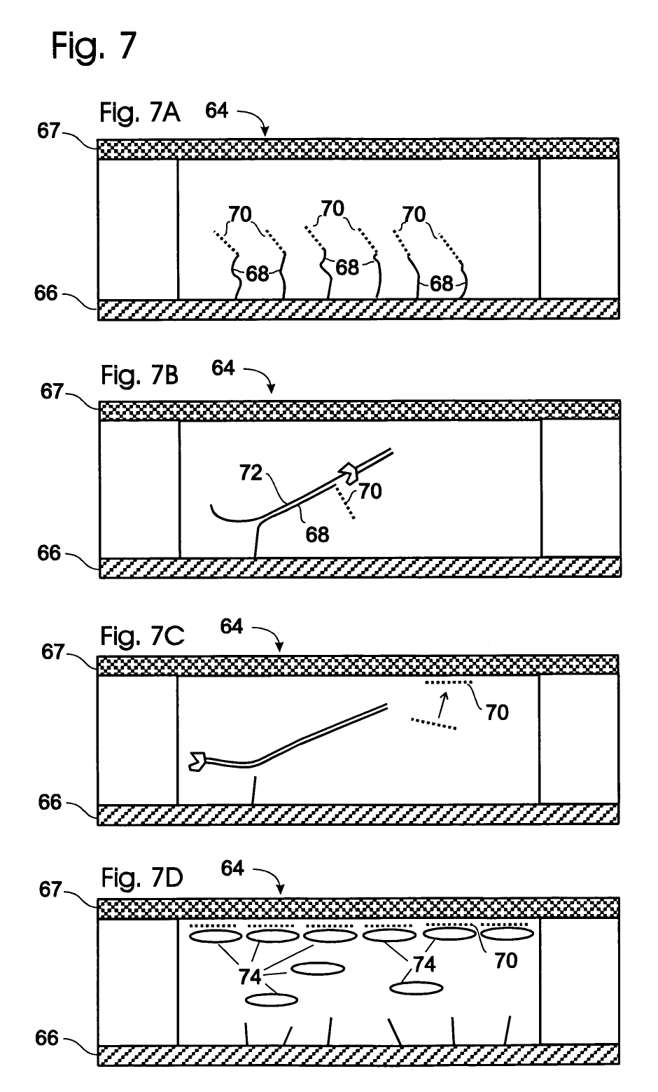

For example, as shown in FIG. 7A, a PCR chamber 64 can be utilized, where the chamber includes an inert solid substrate 66 and an electrode 67 remote from the solid substrate. Multiple nucleic acid strands 58 that are complementary to the desired amplicon can be tethered or otherwise affixed to the solid substrate. The complementary strands can be functionalized by a polyanion moiety 70, where the polyanion moiety can incorporate multiple thiol or disulfide functional groups, or other functional group that exhibits an affinity for binding to gold surfaces.

As shown in FIG. 7B, during amplification, complementary strand 68 hybridizes to amplicon 72, the polyanionic moiety 70 is cleaved from the strand and spontaneously undergoes chemisorption to the gold electrode surface, as shown in FIG. 7C. The subsequent addition of a detection reagent 74 can then be used to transport an electrochemical moiety to the adsorbed thiolated tag sequence, as shown in FIG. 7D.

For example, the electrochemically active group can be incorporated in a hydrophilic dendritic polymer based on poly(ethylene oxide). The dendritic polymer can incorporate a plurality of redox active sites (see Example 6). In another aspect, the electrochemically active moiety can incorporate a plurality of positive charges in order to interact with immobilized nucleic acid sequences via ionic and/or electrostatic interaction (see Example 3).

Selected methods of the invention can be configured for real-time detection (e.g., monitoring of a detection signal over a selected time period, or over multiple amplification cycles, or detecting a signal at a selected point in or after each cycle) or for end-point detection, in which a signal is detected after amplification is complete and compared with an initial or threshold signal to determine the presence, absence, or amount of target polynucleotides. For example, the embodiments illustrated in FIGS. 3 through 7 may be advantageous for real-time detection or for end-point detection, whereas additional embodiments may be more suitable for end-point detection.

Futhermore, although some embodiments are illustrated herein using probes immobilized to a surface, such embodiments can also be adapted for use in solution, such as illustrated in Example 8. Additional guidance for compounds and methods for electrochemical detection can be found in European Patents EP 733058 B and EP 871642 B and PCT Pub. WO 98/20162 (Meade, Kayyam, et al.).

Microfluidics

The methods and materials disclosed herein may be used in conjunction with any of a variety of apparatus or devices. In an advantageous aspect of the invention, the disclosed method can be performed in conjunction with a microfluidic device. A microfluidic device is a device that utilizes small volumes of fluid, on the order of nanoliters, or even picoliters. Microfluidic devices can utilize a variety of microchannels, wells, and/or valves located in various geometries in order to prepare, transport, and/or analyze samples. These microchannels, wells and/or valves can have dimensions ranging from millimeters (mm) to micrometers (μm), or even nanometers (nm). Microfluidic devices may also be referred to as ‘mesoscale’ devices, or ‘micromachined’ devices, without limitation. Microfluidic devices can rely upon a variety of forces to transport fluids through the device, including injection, pumping, applied suction, capillary action, osmotic action, and thermal expansion and contraction, among others. In one example, microfluidic devices can rely upon active electro-osmosis to assist in the transport of aqueous samples, reagents, and buffers. A variety of microfluidic devices are described in U.S. Pat. No. 5,296,375 to Kricka et al. (1994); U.S. Pat. No. 5,498,392 to Wilding et al. (1996); and International Publication No. WO 93/22053 by Wilding et al. (1993); each hereby incorporated by reference.

A microfluidic device useful for detecting a target polynucleotide sequence will typically include a substrate in which a plurality of microfluidic chambers and channels have been fabricated, and a cover adhering to the substrate surface. The device will typically include an inlet configured to receive a sample that contains at least one target polynucleotide sequence, and one or more chambers configured for contacting a probe with the biological sample, wherein the probe comprises a target-complementary segment and a detectable tag as discussed above. The microfluidic device can include one or more chambers configured for subjecting the sample to a polymerase chain reaction, cleaving the detectable tag from the probe, and associating the released tag with a tag complement that is coupled to an electrode to form an immobilized tag:tag complement complex. The tag:tag complement complex is typically detecting and/or quantitated by instrumentation configured for detecting the electrochemical signal related to the presence of the tag:tag complement complex; and the detected/quantitated signal is correlated with the presence/amount of the target polynucleotide sequence in the sample.

A representative microfluidic device, suitable for the amplification and subsequent detection of a target nucleic acid polymer is shown in FIG. 8. The microfluidic device 162 is depicted schematically, and for the sake of simplicity, does not include all the microchannels and wells that may be present in such a microfluidic system. The microfluidic device 162 includes an electrode assembly 164, and a controller 166 configured to control the electrical potential applied at electrode assembly 164. The controller typically serves as both a power supply and instrument for performing amperometric measurements.

Upstream from the electrode assembly 164 is a sample preparation region 168 of the microfluidic device that is configured to prepare a sample solution of interest. Sample preparation region 168 includes reagent reservoirs 170 configured to supply reagents useful for the sample preparation process. The various chambers of the microfluidic device are interconnected via a microfluidic channel system 172 suitable for transporting reagents, sample solutions, and reaction products through the device, and particularly transport such species to and from the electrode assembly 164.

A sample, typically a biological sample, can be introduced into the microfluidic device via an inlet 174. The sample can be introduced by injection, by capillary action, or any other suitable introduction method. The microfluidic device optionally includes a pretreatment well or chamber 176. Pretreatment chamber 176 permits the biological sample to be mixed with reagents for sample digestion, liquidation, or diluting, if desired. Such pretreatment can be used to render the biological sample fluid enough to enhance the effectiveness of downstream processes.

After this pretreatment, the sample can be transported, typically by electro-osmotic pumping, through a filter 178, into a reaction chamber 180. Filter 178 can be used to remove large particles that may interfere with downstream reactions. The filter can be any appropriate filtering agent that is compatible with the biological sample under investigation. For example, filter 178 can include a membrane filter, or a fritted glass filter having a relatively large pore size, for example approximately 100 μm.

Reaction chamber 180 can be used for lysis and denaturing of the sample. As shown in FIG. 8, reagents useful for the lysis and/or denaturing process can be added from reagent reservoir 182 via valve 184. The lysis and/or denaturing process can be accelerated by heating via heating unit 86. Heating unit 86 can include one or more warming lamps, heating coils, fluid heat exchangers, or any other suitable heating apparatus, as well as fans, blowers, heat exchangers, or other suitable cooling mechanism for cooling reaction chamber 180.

After lysis and/or denaturing, the sample is transported to PCR chamber 188. passing through filter 190 en route. Unlike the relatively coarse filter 178, filter 190 is selected for a pore size of approximately 5-10 μm, and is intended to remove undesired byproducts of the lysis/denaturing process. Once the sample has reached PCR chamber 188, reagents useful for the PCR process can be added to PCR chamber 188 from PCR reagent reservoir 192 via valve 194. In an aspect of the invention, the reagents added to the reaction chamber include a probe according to the present invention as discussed above that comprises a segment that is complementary to the target polynucleotide sequence and a cleavable detectable tag. PCR chamber 188 can be heated by heating unit 196. Similar to heating unit 186, heating unit 196 can be any appropriate heating mechanism for facilitating the PCR process, and typically includes a cooling mechanism, so that heat cycling can be accomplished in PCR chamber 188. Selected suitable thermal cycling mechanisms are described in U.S. Pat. No. 5,455,175 to Wittwer et al. (1995) hereby incorporated by reference. It should be appreciated that the PCR chamber can be used in an isothermal mode, for applications that do not require thermal cycling.

After PCR is complete, the sample can be transported to electrolysis chamber 197 through another filter 198 having a pore size of approximately 5-10 μm. Electrolysis chamber 197 includes an electrode 200, controlled by a controller. Although depicted as being electrically connected to controller 166 in FIG. 8, the controller for electrode 200 can be the same or different from the controller for electrode 164. Appropriate reagents can be added to electrolysis chamber 197 from reagent reservoir 202 via valve 204. The tag that is cleaved from the probe by nuclease activity during PCR can then associate with a tag complement that is coupled to electrode 164, where the electrochemical signal related to the presence of the tag:tag complement complex is detected, optionally via the presence of one or more electrochemical mediators.

After amplification is complete, a potential can be imposed between electrode 200 in electrolysis well 197 and electrode 206 in electrolysis well 208. Typically, electrode 200 is held at a cathodic potential, and electrode 206 is held at an anodic potential so that, in conjunction with a thin layer of crosslinked polyacrylamide gel 208, electrophoresis occurs across gel 210. While electrophoresis is occurring, electrode 164 is typically electrically neutral.

The polyacrylamide gel is typically prepared with a low degree of crosslinking. Under these electrophoretic conditions, all nucleic acid fragments with the exception of DNA that has complexed and hybridized will migrate to electrolysis chamber 210. Relatively large nucleic acid complexes are left behind due to their large size and relative inability to penetrate the thin layer of crosslinked polyacrylamide gel.

Although electrophoretic separation has been described, any suitable separation process could be used to isolate the cleaved tag, including for example, mechanical separation, size exclusion chromatography, separation using derivatized beads or matrix, for example including magnetic beads or a streptavidin-modified matrix.

Once the tag:tag complement complex is associated with the electrode surface, the detectable label present in the complex may be detected and/or quantified, as discussed above, and in the following examples.

Kits

The probes disclosed herein may be provided in the form of kits for detecting a target polynucleotide sequence, according to the methods of the invention. These kits optionally include one or more probes that include a segment that is complementary with a selected target polynucleotide sequence, and a detectable tag. The kit can include probes that are selective for a plurality of independent and distinct target polynucleotide sequences. The kit can include probes that each have a distinct and individually detectable tag. The kit optionally includes one or more tag complements for forming tag:tag complement complexes upon cleavage of the tag from the probe. The kit optionally includes samples of target polynucleotide sequences corresponding to probes present in the kit, for example for the purpose of calibration. The kit optionally includes one or more buffers or buffering agents suitable for preparing solutions of the probe and/or solutions of the target polynucleotide sequence.

The kit optionally incorporates additional reagents, including but not limited to electrochemical calibration standards, enzymes, enzyme substrates, nucleic acid stains, labeled antibodies, and/or other additional detection reagents. The probes of the invention optionally can be present as a lyophilized solid, or as a concentrated stock solution, or in a prediluted solution ready for use in the appropriate assay. Typically, the kit is designed to be compatible for use in an automated and/or high-throughput assay, and so is designed to be fully compatible with microfluidic methods and/or other automated high-throughput methods.

Electrochemical Compositions

The invention also provides electrochemical compounds such as are described herein. Such compounds are useful for a variety of electrochemical applications, including but not limited to the methods of detecting a target polynucleotide sequence disclosed herein.

For example, the present disclosure provides bis-osmium compounds of the form shown in Scheme 5, below.

Also disclosed herein are compounds of the form shown in Scheme 10 at compound 21 and the structurally related compounds that can be produced using the alternative aromatic alkyl amines reactants illustrate in Scheme 10.

Also disclosed are poly-osmium compounds of the form shown in Scheme 6 (e.g., compounds 10 and 11), Scheme 7 (e.g., compounds 14 and 15), Scheme 15 (e.g., compound 2), Scheme 16 (e.g., compound 4), Scheme 17 (e.g., compound 6), and Schemes 18 through 20, and analogs and derivatives thereof.

### EXAMPLES

### Example 1

### Preparation of a DNA Amplification Probe

A probe is prepared that is selective for amplification of the listeriolysin (Hly) gene of the food pathogen Listeria monocytogenes. The complementary probe (or primer) is biotinylated at the 5′-end of the sequence. The complementary probe also contains biotin at the last dT residue, as well as a 3′-terminal amino group to prevent elongation of the probe during PCR. The complementary sequence is modified by a 19-base tag sequence shown in bold below.

5′-
CACGAATCAAAGCTCTCAACGCCTGCAAGTCCT*AAGACGCCA-3′NH₂

where T* marks the biotinylated base. Biotinylation permits removal of the complementary sequence using streptavidin-modified beads.

The presence of the tag sequence does not influence PCR amplification of the target sequence, as verified by electrophoretic analysis of the amplicon compared to control reactions.

The forward and reverse primers used during the PCR are as follows:

5′-CATGGCACCACCAGCATCT
and
5′-ATCCGCGTGTTTCTTTTCGA

where the 5′-terminus of each primer is also biotinylated, for removal using streptavidin beads.

The PCR reaction was run for 10 min at 95° C., then (15 sec. at 95° C., 1 min at 63° C.)×40 cycles in PCR buffer A (Applied Biosystems, Ca # N808-0228) supplied with 6 mM MgCl₂. Primers and probe were at concentrations of 200 nM and 400 nM, respectively. The HPLC column XTerroMSC18 (2.5 mm×50 mm) from Waters Corp. was equilibrated with 7% ACN (acetonitrile)+93% TEAA (0.1 M triethanolamine acetate, pH 6.8). A gradient elution (0.3 ml/min, 60C) was performed in three steps: Step 1: 7% ACN+93% TEAA for 7 min. Step 2: 10% ACN+90% TEAA for 10 min. Step 3: 35% ACN+65% TEAA for 10 min. (ACN—Acetonitrile. TEAA—0.1M Triethanolamine—Acetic acid at pH 6.8). PCR is run for 10 minutes at 95° C., then (15 sec. at 95° C., 1 min. at 63° C.)×40 cycles, at concentrations of 200 nM primers and 400 nM probes, respectively.

After completion of the polymerase chain reaction (PCR), the reaction mixture is adjusted to 1 M salt by addition of NaCl solution. The reaction mixture is then incubated with streptavidin-coated magnetic beads for 15 minutes. The biotinylated complementary probes, including complementary probes that still include uncleaved tag sequences, are adsorbed to the magnetic beads and removed from the reaction mixture. Biotinylated amplicon is similarly removed from the mixture, leaving cleaved tag sequences in the solution.

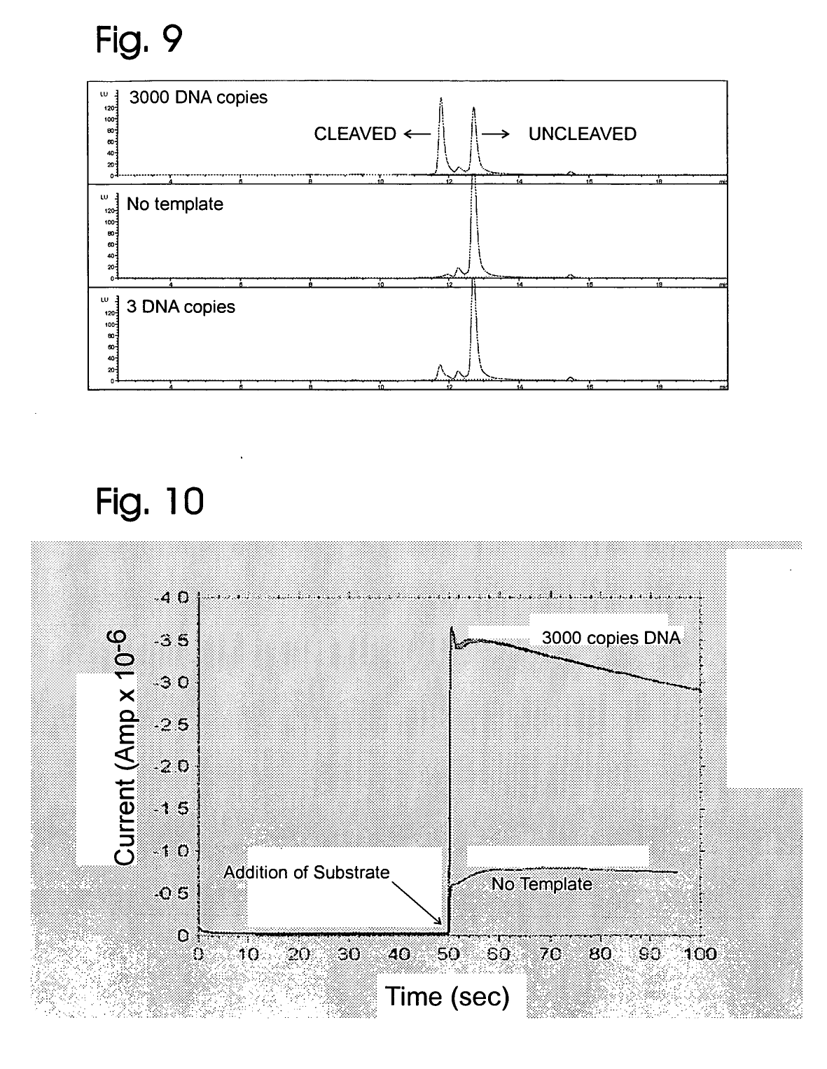

Amplification and cleavage carried out with tag sequence that is labeled on the 5′-terminus with a fluorescent label (fluorescein), followed by characterization by HPLC, shows that a cleavage product 20 bases in length is produced, and that depletion of the reaction mixture with streptavidin-coated beads removes noncleaved probe. As shown in FIG. 9, about 50% of probe is cleaved at 3000 copies starting material of Listeria DNA., whereas no template control does not contain cleaved oligonucleotide. this method is sensitive enough, however, to produce detectable cleavage product after generating at 3 copies of template in the reaction mix.

The tag sequence can also be detected electrochemically. After depletion on streptavidin-coated beads, the target-containing sample and a no template control solution are both exposed to separate gold electrodes functionalized with an immobilized capture probe complementary to the cleaved sequence. The solutions are allowed to hybridize for 1 hr at 45° C. directly from the bead separation step, and then rinsed with 10 mM Tris 100 mM NaCl (pH=8). The electrodes are then exposed to a 100 μg/mL solution of a threading intercalator labeled with electrocatalytic osmium 2,2-bis(bipyridine) (see the structure in Scheme 1) in 10 mM Tris, 100 mM NaCl. (pH=8) in the electrochemical cell for 5 min. After washing with PBS buffer (20 mM phosphate and 100 mM sodium chloride pH 7) and a phosphate buffer saturated with NaCl in 10% ethanol, a baseline current is obtained in 200 μL PBS at 0.2 V vs Ag/AgCl. Upon addition of the 800 μL 6.25 mM ascorbic acid substrate, the current increased in proportion to the amount of intercalator and thus the hybridized target (see FIG. 10).

Electrodes and Electrochemical Apparatus. Working electrodes are fabricated by blanket sputter coating 4″ silicon wafers with a 100 angstrom Cr layer followed by a 2000 angstrom gold layer (Lance Goddard Associates, Foster City, Calif.). The wafers are then diced by hand to form segments approximately 1 cm×1.5 cm. The electrodes are cleaned using a UV-Ozone cleaner (Model 42, Jelight Company, Inc, Irvine, Calif.) for 20 minutes, followed by exposure to absolute ethanol for 20 min. The electrodes are then exposed to a 0.5 μM solution of a thiolated DNA capture probe in 1 M potassium phosphate buffer (pH=7) for 10 minutes followed by a 5 sec water rinse. The capture probe sequence is shown immediately below:

5′ (DTPA)(DTPA)(DTPA) AAA AAA TTG AGA GCT TTG ATT
CGT G 3′

where DTPA is a disulfide-containing phosphate linker of the type shown in Scheme 20, prepared from dithiol phosphoramidite (Glen Research, Sterling, Va.). The electrodes are then exposed overnight to a 1 mM solution of mercaptohexanol in water followed by a 30 sec water rinse. The electrodes are then dried under nitrogen.

Electrochemical measurements can be performed in an electrochemical cell with a ⅛″ ID o-ring defining the working electrode area vs. a Ag/AgCl reference electrode (Cypress Systems, Lawrence, Kans.) and a platinum coil counter electrode using a CHI model 660B potentiostat (CH Instruments, Austin, Tex.).

### Example 2

### Electrochemical Monitoring of PCR Progression Using Ferrocene (Fc) Labeled Probe

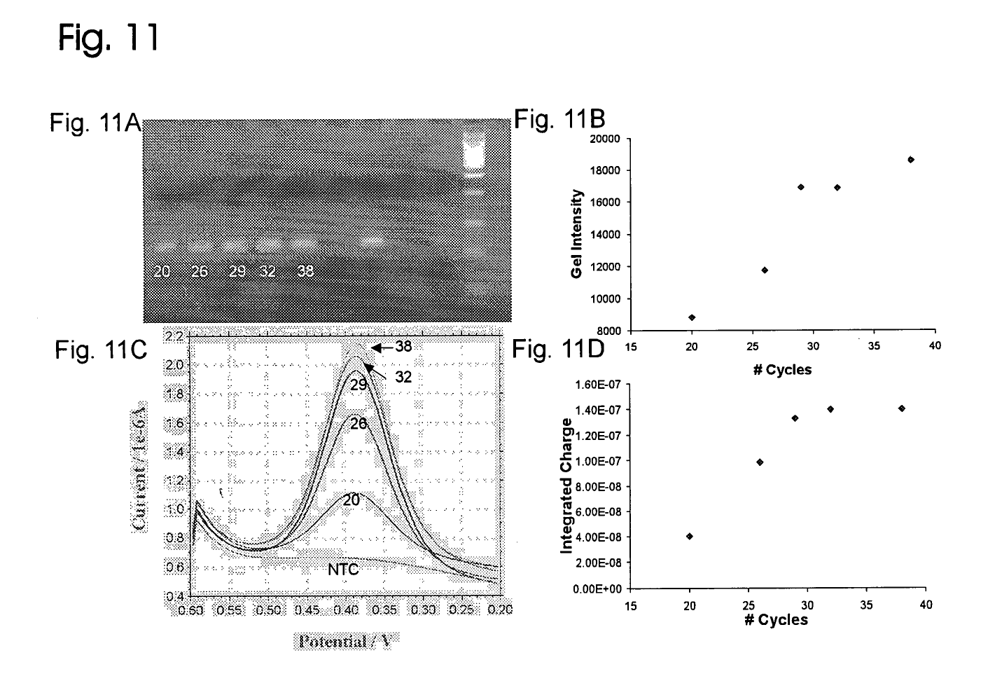

In this experiment, the composition of the reaction mixture and amplification protocol were the same as in Example 1, except that the probe was substituted by a ferrocene moiety at its 5′ end (“Synthegene”, Houston, Tex.) and 100,000 copies of Listeria DNA was used as a template. Six tubes containing 50 μl aliquots of identical PCR reaction mixtures were placed into 9700 thermocycler (Applied Biosystems, Foster City, Calif.). Tubes were removed from the thermocycler sequentially after 20, 26, 29, 32 and 38 cycles of amplification. The tube corresponding to no template control (NTC) was removed after 38 cycles. The cleaved, ferrocene-containing 20-mer fragment (a detectable tag) was purified from uncleaved probe using streptavidin magnetic beads as described in Example 1. 30 μl aliquots of purified Fe 20-mers (in 1 M NaCl) were placed on the surface of a gold electrode for 1 h to allow hybridization with complementary capture oligonucleotide. After brief rinsing of electrodes with PBS buffer, each electrode was placed into the chamber described in Example 1. Chamber was filled with approx. 100 μl of PBS buffer and electrochemical measurements were made as shown in FIG. 11.

Electrochemical signal amplitude is dependent upon the number of PCR cycles performed. FIG. 11A shows the results of gel electrophoresis analysis of amplicons and densitometric quantitation. FIG. 11B is a plot of the amounts of amplicons vs. number of PCR cycles. FIG. 11C demonstrates results of electrochemical measurements. FIG. 11D shows a plot of electrochemical signal values (areas of peaks) vs. number of PCR cycles. The correspondence of the curves shown in FIGS. 11B and 11D indicates that this methodology allows quantitative monitoring of PCR reactions.

### Example 3

### Preparation of an Electrocatalytic Nucleic Acid Intercalator

The detection reagent is optionally an electrochemical moiety that is an intercalator for nucleic acid strands. An exemplary intercalator has the formula shown in Scheme 1. 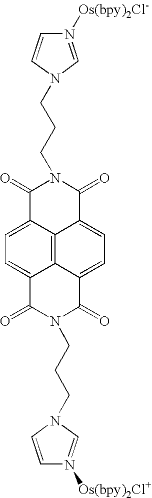 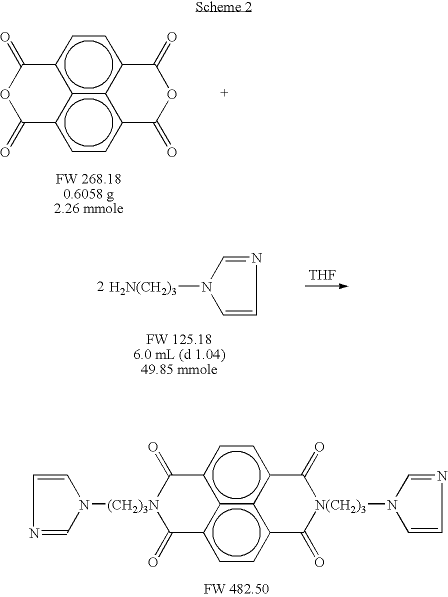
and is prepared similarly to the method of Tansil, et al. (Anal. Chem. 2005, 77(1), 126-134). A solution of 6.0 mL (24.92 mmole) of 1(3-aminopropyl)imidazole in 3.0 mL of anhydrous THF is charged into a 50 mL two-necked round-bottomed flask that is equipped with a water-cooled condenser, a pressure-equalizing addition funnel, and a ¼″ magnetic stir bar. The reaction scheme is provided in Scheme 2.

To this solution is added a suspension of 0.6058 g (2.26 mmole) of 1,4,5,8-naphthalene tetracarboxylic dianhydride in 3.0 mL of anhydrous THF, over a period of 15 minutes with constant stirring. The reaction mixture is heated at reflux for 24 hours. The color of the reaction mixture turns from light yellowish orange to very dark brown within 30 minutes. At the end of the reaction time, the reaction mixture is cooled to ambient temperature and 20 mL of a mixture of acetone/water (3:1 v:v) is added with rapid stirring. The mixture is allowed to stand at ambient temperature for 5 minutes. The supernatant layer is decanted. To the residue is added 10 mL of methanol and the resulting slurry is stirred. The yellow crystals are collected by suction filtration. The filter cake is washed briefly with methanol and air-dried with suction. The precipitate is recrystallized from 20 mL chloroform/ethanol mixture (1:1 v:v), followed by vacuum drying at 40° C. overnight to give 0.236 g (22% yield, reported yield 85%) of product. The ¹H-NMR spectrum of the product agrees perfectly with that reported in the literature. A scheme for this reaction is provided in Scheme 3.

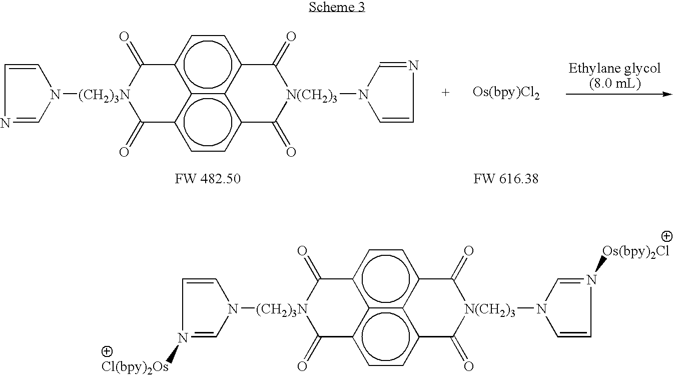

To a solution of 0.642 g (0.52 mmol) of OS(bpy)₂Cl₂ in 16.0 mL of ethylene glycol, 0.236 g (0.25 mmol) of PIND (see structure on left side of Scheme 3) is added in smalls portions over a period of 10 minutes with constant stirring. The final mixture is heated at an oil bath temperature of 180° C. for 30-40 minutes. The ethylene glycol is removed by rotary evaporation at 60° C. to give a viscous oily residue. To the viscous residue is added 150 mL of THF with vigorous stirring, with formation of a resulting precipitate. The precipitate is collected by suction filtration, rinsed with anhydrous THF, and dried on the filter to give 129.3 mg (50% yield, reported yield 78%) of product as a dark purple powder that is very soluble in water and ethanol. In contrast, the starting material (OS(bpy)₂Cl₂ is a dark purple powder that is insoluble in water and barely soluble in ethanol. The UV-visible spectrum of the product agrees with that reported in the literature.

### Example 4

### Polycationic Electrochemical Moieties

In selected aspects of the disclosed method, a tag sequence is immobilized at a remote electrode before detection. For example where the tag sequence includes one or more sulfur-containing functional groups such as thiols or disulfides, and the electrode includes a gold metal surface. In these aspects, detection of the tag sequence can be facilitated by the addition of an electrochemical moiety that interacts electrostatically with the adsorbed tag sequence. Such electrostatic interactions do not rely on or require hybridization of a surface polynucleotide probe with the tag sequence.

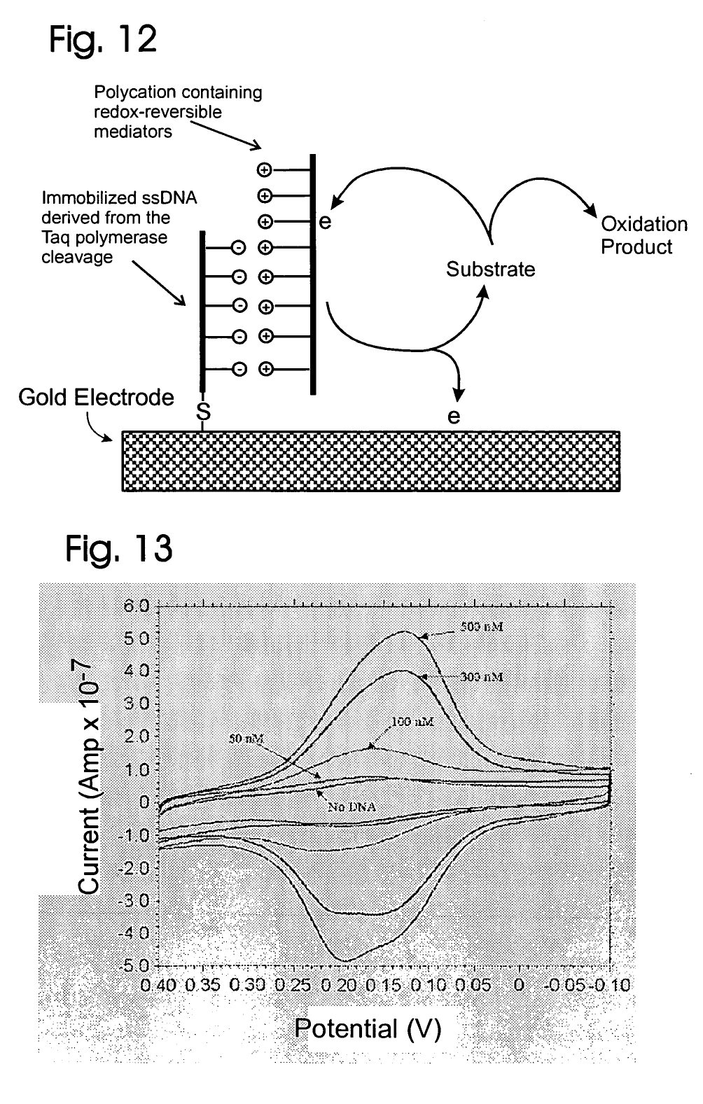

The binding of a polycation to a thiolated bound tag sequence is shown in a simplified diagram below in Scheme 4: 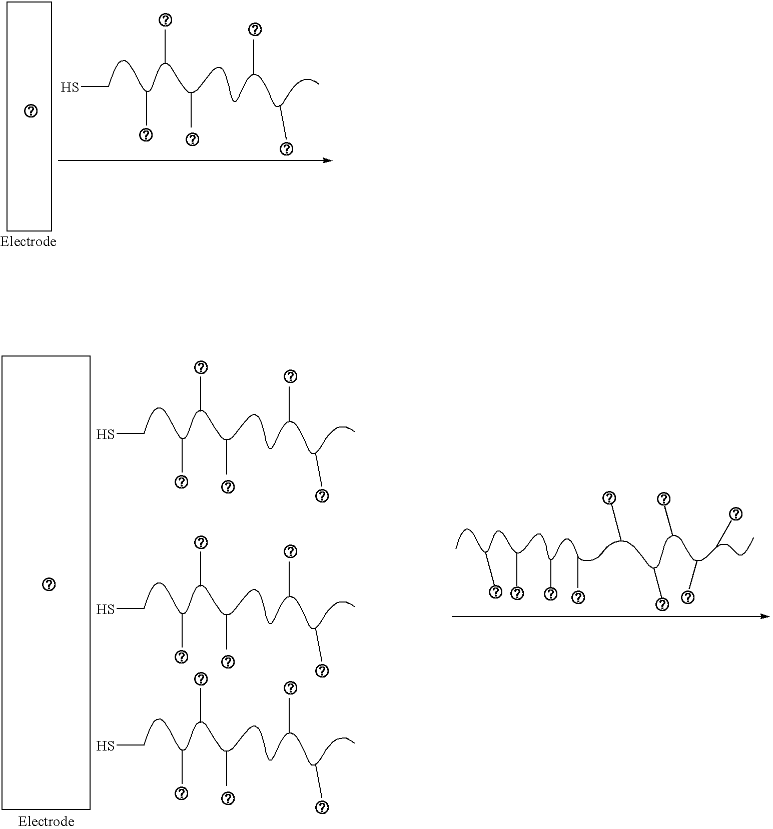 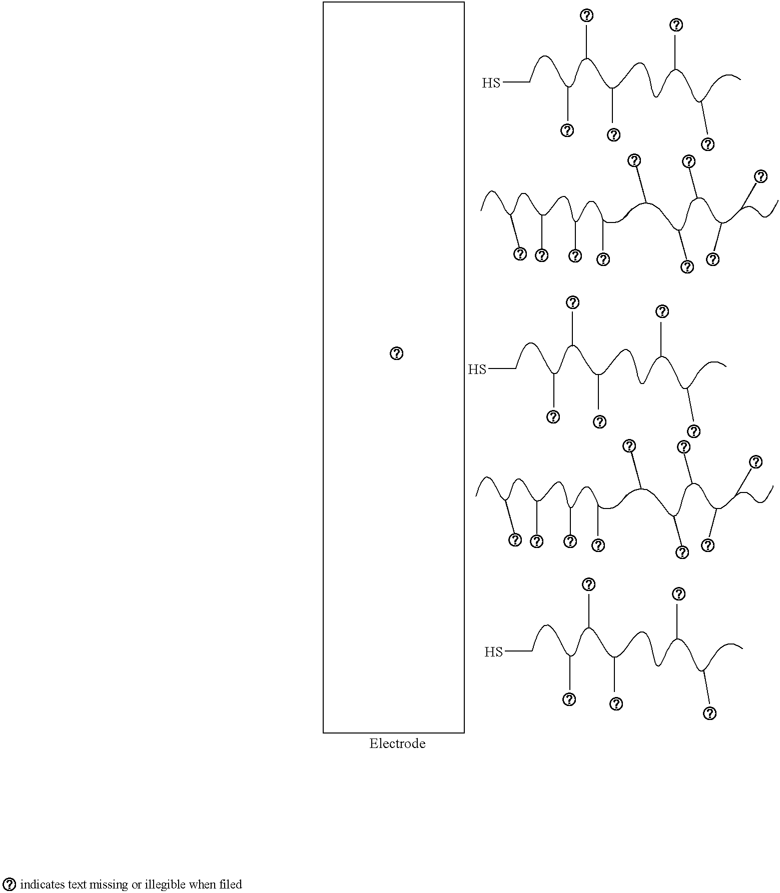
where the polycation includes a plurality of redox reversible centers. The electrostatically bound redox centers can mediate detection at the electrode surface, as shown in FIG. 12. Note that the ‘substrate’ of FIG. 12 can refer to any redox active compound or material that can facilitate detection of the detectable tag. (here, the bound tag is a ssDNA flap which is polyanionic, and which is complexed with a polycation moiety that contains redox active moieties (e.g., osmium complexes) whose presence can be detected by the redox cycle shown in FIG. 12).

### Example 3A

The redox reversible polycation can include osmium complexes of α,ω-diimidazolylalkanes, as shown below in Scheme 5.

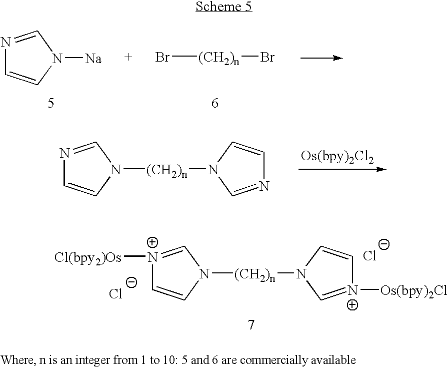

### Example 3B

Alternatively, the polycationic electrochemical moieties can include additional osmium complexes, as shown below. The bis- and tetra-osmium complexes are synthesized by reacting the appropriate diacid or tetraacid, respectively, with thionyl chloride. The resulting acyl chloride compound can be purified by vacuum distillation, among other methods. In some aspects, the acyl chloride compounds are converted to their N-hydroxysuccinimide (NHS) ester counterparts. The NHS esters can be prepared by treating the acids with disuccinimidyl carbonate (DSC) in the presence of diisopropylethylamine (DIPEA). The reaction strategy is shown in Scheme 6.

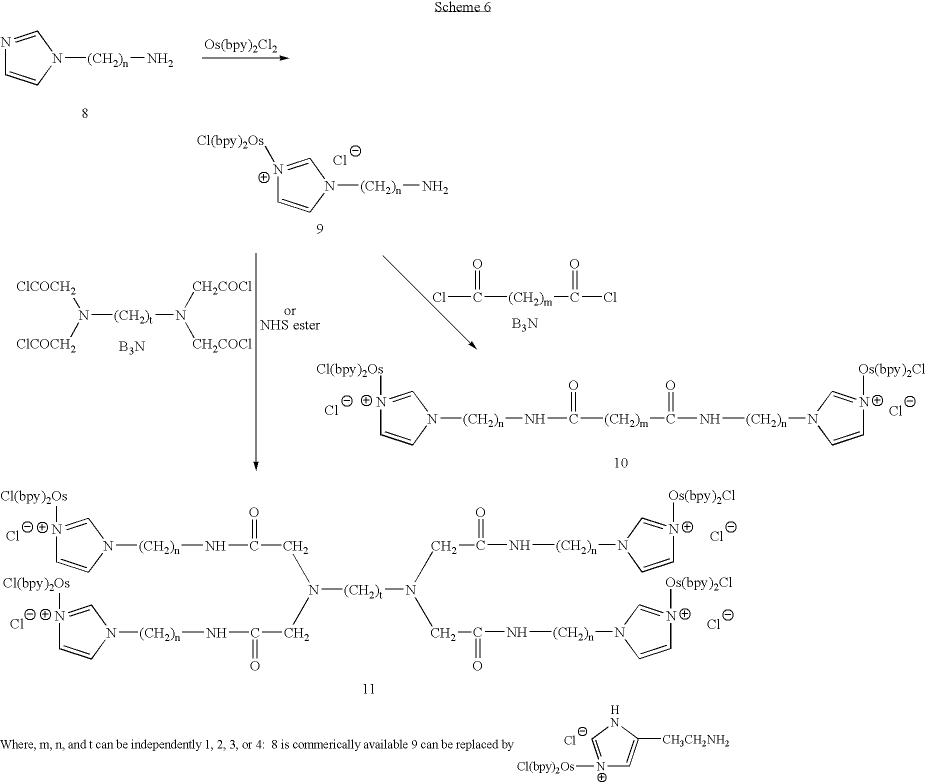

### Example 3C

Alternatively, bis- or tetra-osmium complexes can be prepared according to the protocol shown below. The reaction of 2-(2-aminoethyl)pyridine and Os(bpy)₂Cl₂ in aqueous ethanol, with precipitation and purification of the product yields the desired osmium complex. The di- and tetra-acyl chloride compounds can be prepared according to the protocol described above, including their NHS ester analogs. The synthetic strategy is shown in Scheme 7.

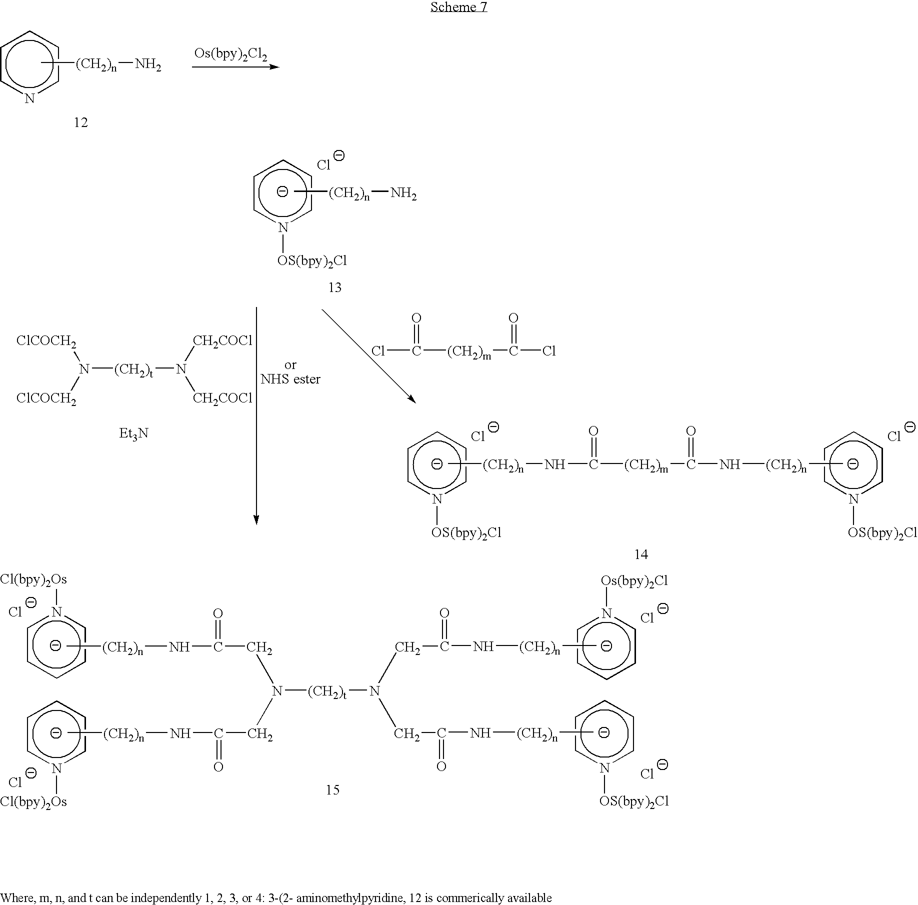

### Example 3D

In yet another rex ample, the redox reversible polycation can be a polymer that includes a plurality of electroactive centers, for example such as osmium complexes. Such a polymeric polycation can be prepared by treating poly(4-vinylpyridine) and Os(bpy)₂Cl₂ according to a protocol similar to that reported in U.S. Pat. No. 5,262,035 (hereby incorporated by reference). In order to prevent α-elimination at high pH, the 2-amino- or 2-hydroxyethyl group attaching to the pyridinium ring system can be substituted with a methyl group. The synthetic strategy is shown in Scheme 8.

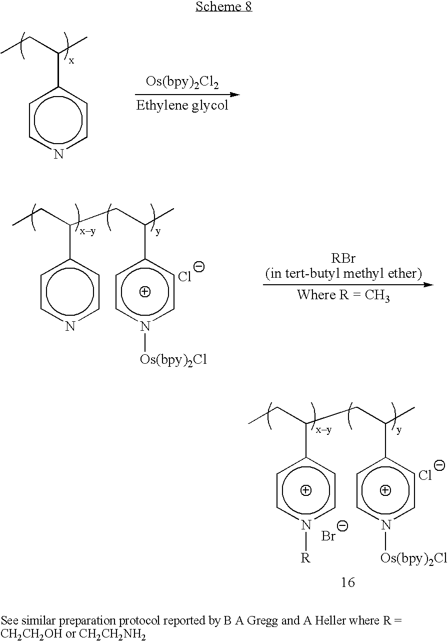

### Example 3E

In selected alternative embodiments, the polycationic electrochemical moiety can be prepared from poly(1-vinylimidazole), as shown below. The polymer backbone can be prepared by solution polymerization of 1-vinylimidazole using ammonium persulfate as an initiator in the presence of TEMED. The free radical polymerization of the 1-vinylimidazole can also be initiated by a water-soluble azo-compound, for example 2,2′-azobis[2-(2-imidazolin-2-yl)propane]dihydrochloride. The resulting polymer 18 is water soluble and can be purified, for example, by dialysis. Treatment of the polyimidazole compound with, for example, Os(bpy)₂Cl₂ in aqueous ethanol results in the addition of multiple redox active centers on the polymer. At high degrees of conversion, where few imidazole rings remain unsubstituted, the resulting polycationic compound can be isolated and purified by precipitation from THF. In some aspects, a desired monomer, such as N,N-dimethylacrylamide can be copolymerized with the vinylimidazole compound, in order to permit the specific physical and chemical properties of the resulting polymer to be varied as desired. The synthetic strategy is shown in Scheme 9.

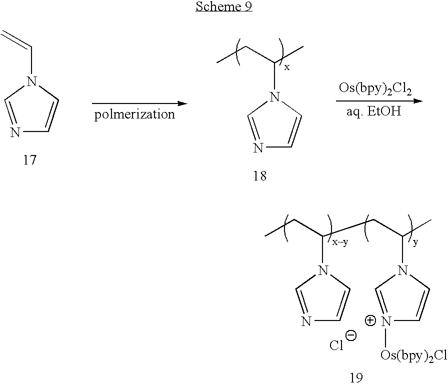

### Example 3F

In selected embodiments, the polycationic electrochemical moiety can include a water-soluble polymer having hydroxyazetidinium groups along the polymer backbone, prepared by polymerization or copolymerization of N,N-diallyl-3-hydroxyazetidinium salt. Alternatively, POLYCUP polymer can be prepared by treating epichlorohydrin with a polyamide of adipic acid and N,N-di(2-aminoethyl)amine. polymer 20 is highly cationic and very soluble in water. The azetidinium chloride is highly reactive to amino and carboxylic groups. Redox reversible imidazolyl-Os(bpy)₂Cl can be incorporated as shown to give 21, which is isolated by precipitation from methanol or THF, and can be purified by dialysis.

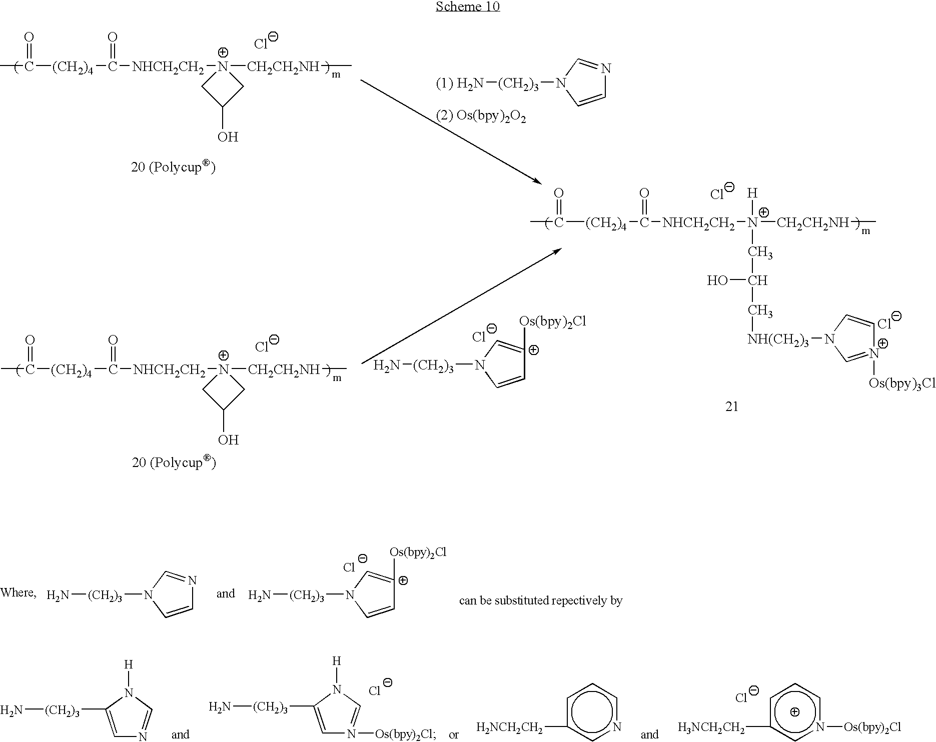

### Example 3C

### Alternatively, rather than osmium complexes, electroactive ferrocene complexes can be incorporated by reaction of 20 with 2-amino ethyl ferrocene or ferrocenacetic acid.

### Example 3D

In a particular example, an aliquot of 12% solution of POLYCUP polymer 20 is added to an equal amount of 10% aqueous solution of poly (ethyleneimine) 24 a multi-functional amine. The reaction mixture turns into a solid gel within 15 minutes of heating in a water bath at 50° C., indicating the high reactivity of azetidinium rings in 20 towards amine functional groups. No gel is formed when 24, a multi-functional amine, is replaced by a 3-aminopropylimidazole 25, a mono-functional amine, for reaction with 20. See Scheme 11.

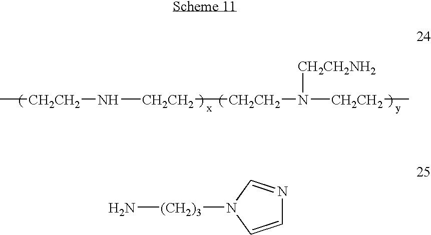

### Example 5

### Preparation of 1,1′-(1,6-hexanediyl)bis(imidazole)

Into a 250 mL three-necked round-bottom flask, equipped with a magnetic stirrer, an addition funnel, and a Dean-Stark condenser, 3.0 mL of water and 5.82 g (103.7 mmol) of potassium hydroxide were charged with stirring. Upon dissolution of the KOH, 100 mL of toluene is added, followed by addition of 6.81 g (100.0 mmol) imidazole. Upon the dissolution of imidazole, 40.0 mL of DMSO is added.

The mixture is heated and stirred in an oil bath at 133° C. until no more water is codistilled out, or approximately 4 hours. About 8 mL of water is generated.

After the temperature of the reaction mixture drops to below 90° C., 12.07 g (49.5 mmol) of 1,6-dibromohexane is added dropwise with constant stirring. A white precipitate is formed during the addition.

The mixture is stirred at 85-90° C. for 17 hours.

The potassium bromide is removed by filtration. The toluene and DMSO in the filtrate is removed by distillation under reduced pressure (49° C., 0.5 mm Hg) to yield a viscous oil.

A small amount of white crystalline solid is vacuum distilled (90° C. at 0.02 mm Hg) and identified as imidazole by ¹H-NMR spectroscopy. The residual oil is subjected to chromatographic purification using a mixture of 1:1 v:v dichloromethane and methanol to give 8.0 g (74% yield) of product. The ¹H-NMR spectrum of the product agrees with the expected structure. The synthetic scheme is shown in Scheme 12.

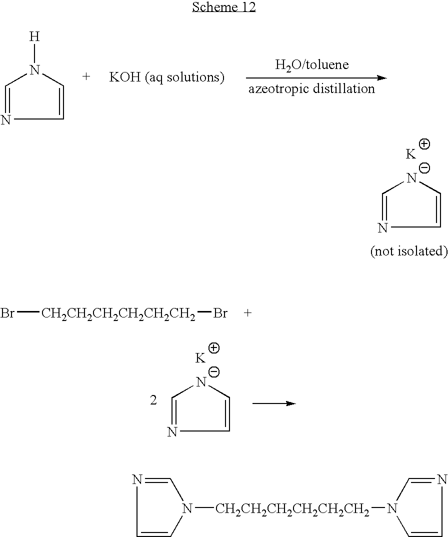

### Example 6

### Preparation of osmium-substituted 1,1′-(1,6-hexanediyl)bis(imidazole)

Into a 500 mL round-bottom flask, equipped with a magnetic stirrer and a condenser, 2.0 g (3.5 mmol) of osmium bipyridinyldichloride, 0.38 g (1.76 mmol) of 1,6-bis(imidazolyl)hexane, 100.0 mL of ethanol, and 100.0 mL of deionized water is added. The reaction mixture is heated to a gentle reflux in an oil bath for 16 hours to give a dark purple solution. The reaction mixture is then cooled to ambient temperature, and the solvent is removed under reduced pressure.

The residue is triturated in 60 mL of THF for 15 minutes. The precipitates are collected by suction filtration, rinsed with THF to remove 1,6-bis(imidazolyl)hexane, and suction air dried to give a dark purple crystalline powder.

The product is vacuum dried at 55° C. overnight to give 2.26 g (94.5 % yield) of product. The ¹H-NMR spectrum of the product agrees with the expected structure. The synthetic scheme is shown in Scheme 13.

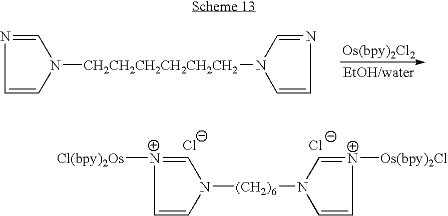

### Example 7

### Response of Compound 7 at DNA-modified Electrodes

Planar gold electrodes are fabricated by blanket sputter coating a silicon oxide wafer with a 10 nm chrome layer, followed by a 500 nm gold layer. Scribed fragments of this wafer are then cleaned using a commercial UV-Ozone cleaner for 30 minutes, followed by soaking for 20 minutes in absolute ethanol. The electrodes are then dried under nitrogen. Using a PDMS gasket to define the exposure area, varying concentrations of a DTPA-modified 25-base DNA strand (where DTPA is a disulfide-containing phosphate linker of the type shown in Scheme 20) are deposited on the electrode surface for 20 minutes, followed by a 10 minute exposure to 1 mM mercaptohexanol in water. After rinsing with water, the electrodes are then exposed to a 100 μg/mL solution of 7 (see Scheme 14) in Millipore water for 1 minute, and rinsed for 20 seconds in water.

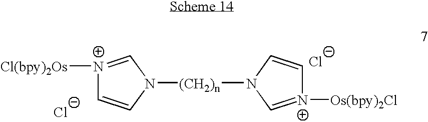

The electrodes are then fitted into an electrochemical cell with a 3 mm diameter o-ring to define the electrode area. Cyclic voltammograms are performed using the electrode at 100 mV/sec in a 10 mM Tris buffer (pH 8) using a platinum counter electrode and a Ag/AgCl reference electrode, as shown in FIG. 13.

The measured integrated charge is then plotted versus DNA concentration for compounds 1 and 7. The resulting plot is shown in FIG. 14.

### Example 8

### Dendritic Electrochemical Moieties

Redox reversible mediators can be prepared via a four-armed poly(ethylene oxide) succinimidyl terminated pentaerythritol, commercially available through Polymer Source (Quebec, Canada). As illustrated below, the reaction of the erythritol intermediate gives a compound that then reacts with Os(bpy)₂)Cl₂ to give a four-armed redox moiety. The resulting compound is hydrophilic, and less susceptible to chemisorption. For each polycation shown herein, the counterion can be replaced via any suitable ion-exchange method, for example anion exchange resin, or dialysis at pH greater than 7.

A synthetic strategy for the preparation of a four-armed electrochemical moiety is shown in Scheme 15.

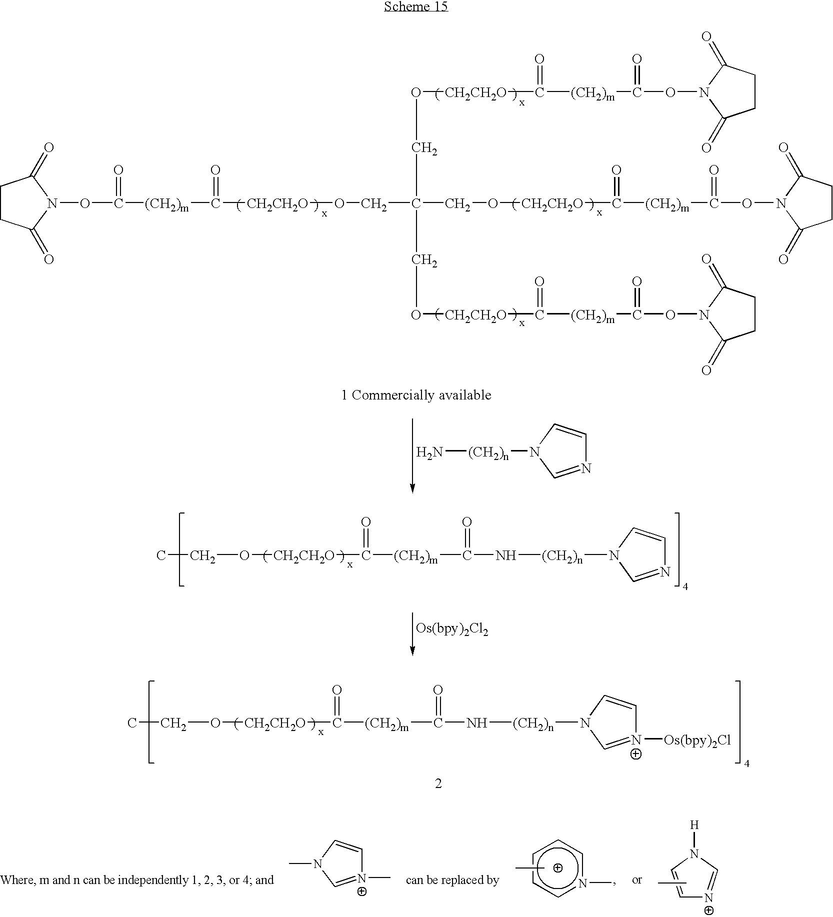

Redox reversible mediators can also be prepared with additional numbers of arms. For example, a six-armed moiety can be similarly prepared with a six-armed poly(ethylene oxide) succinimidyl-terminated dipentaerythritol that is also commercially available (Polymer Source, Quebec, Canada). As shown in Scheme 16, the reaction of the succinimidyl ester compound with an amine-containing complex of transition metal yields a six-armed dendritic mediator.

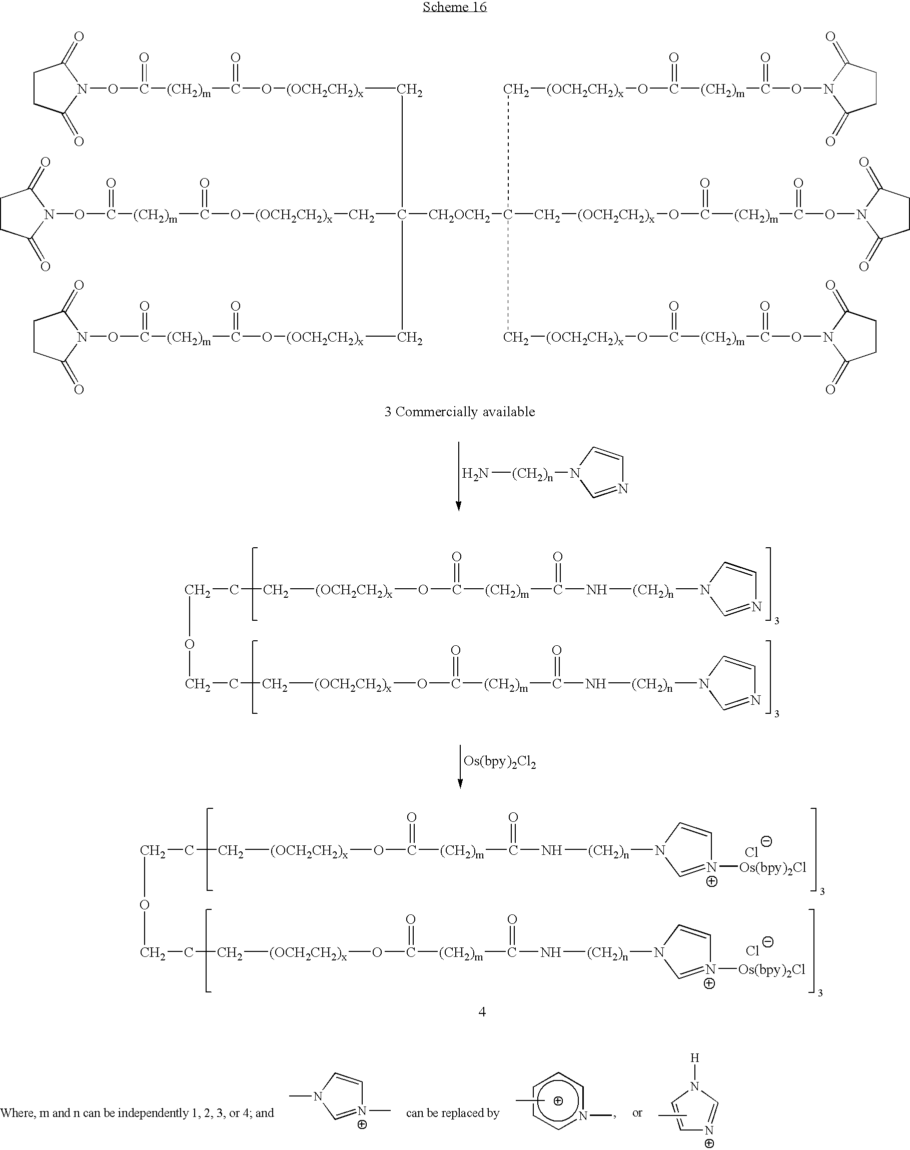

An alternative example of a six-armed dendritic electrochemical mediator can be prepared as set out below in Scheme 17, where a six-armed poly(ethylene oxide) succinimidyl terminated trimethylolpropane is used to prepare a six-armed dendritic mediator that includes osmium-based redox centers.

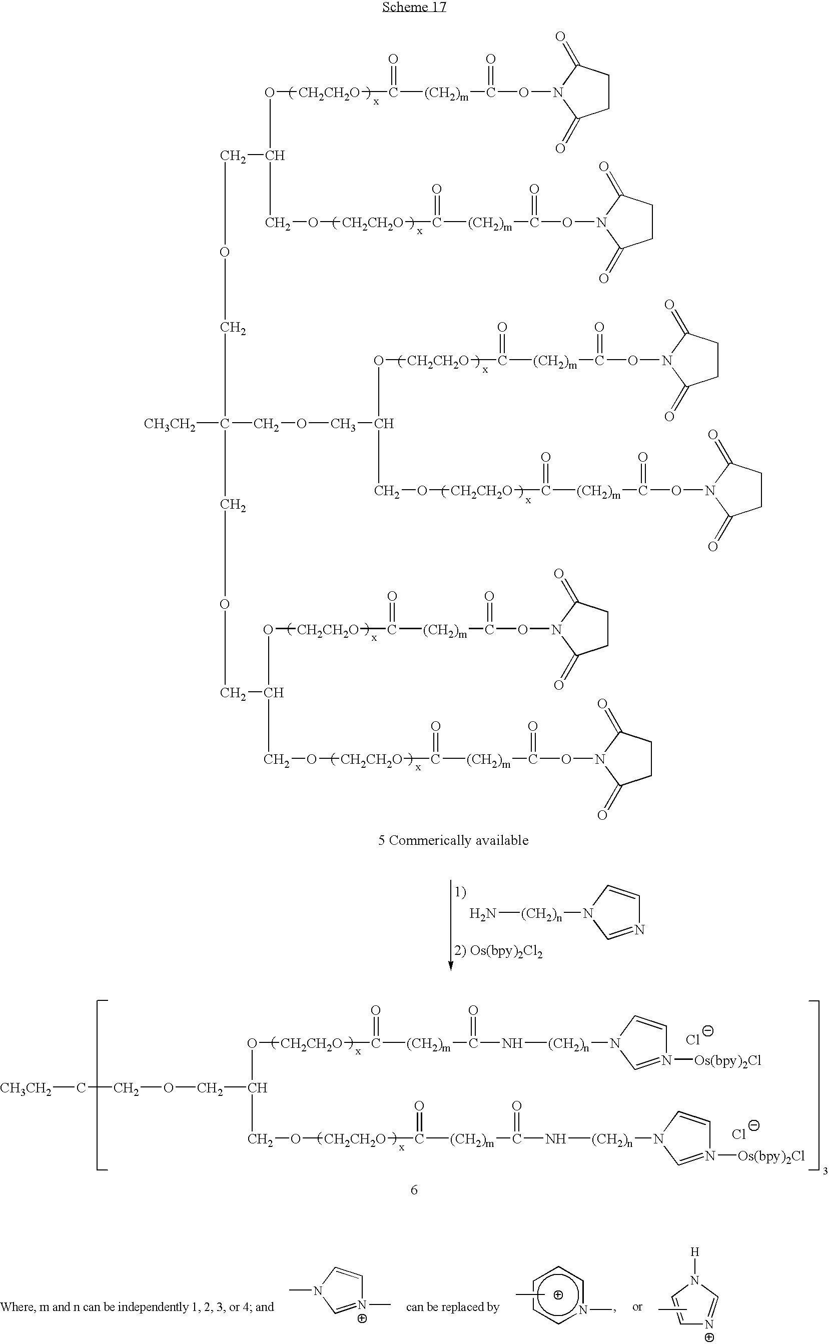

### Example 9

### Preparation of Detection Tags Including Osmium Redox Centers

Detection tags can be prepared that include an osmium electroactive moiety according to the synthetic strategy of Scheme 18.

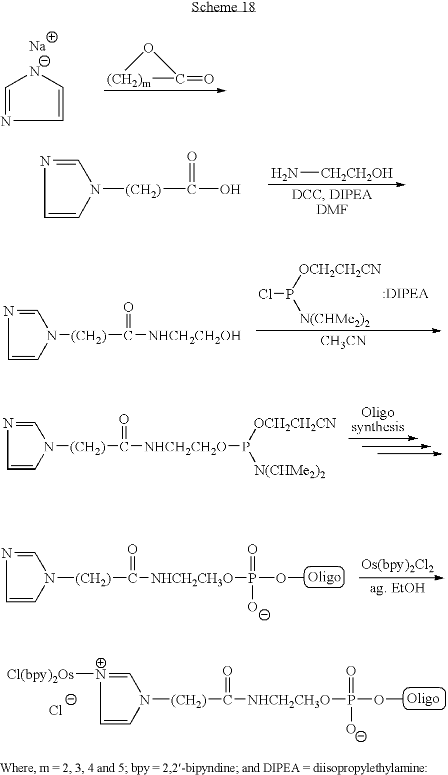

As an alternative to the imidazole-based detection tag, detection tags that include an osmium center can also be prepared that incorporate a pyridine ring, as shown in the synthetic strategy shown in Scheme 19.

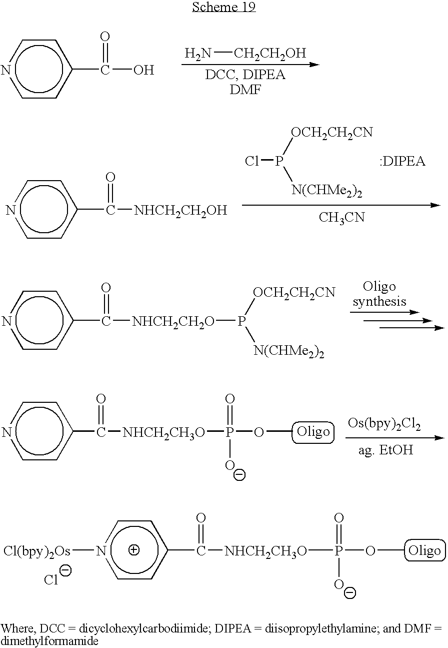

A detection tag incorporating both an osmium redox center and a disulfide moiety is prepared as shown in the synthetic strategy of Scheme 50.

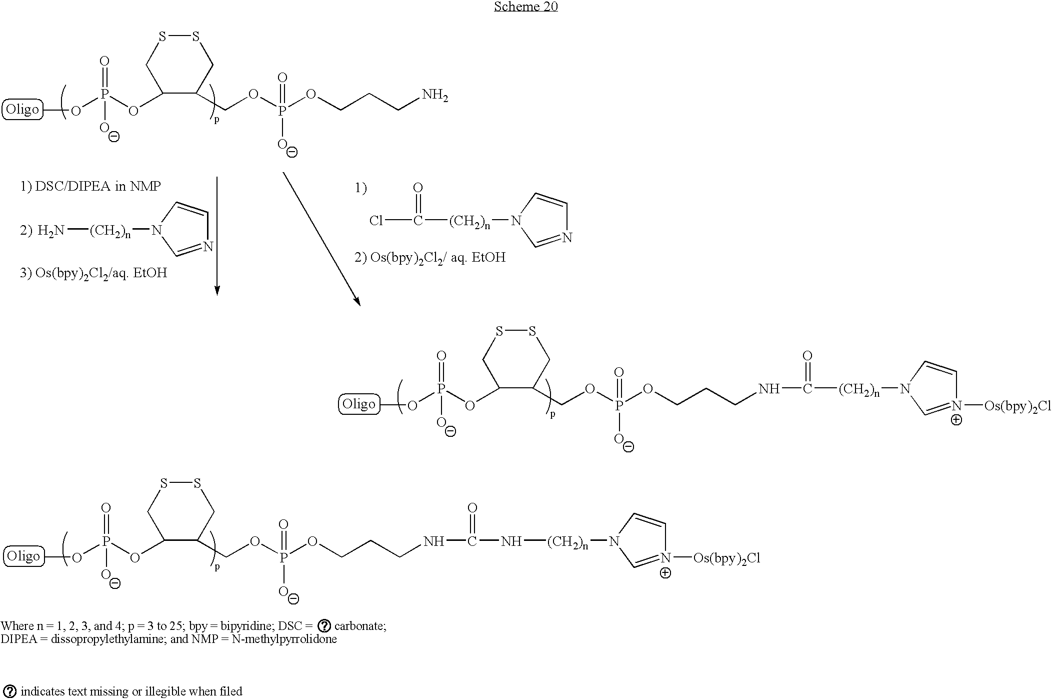

The disulfide-labeled oligonucleotide starting material is itself useful as a detection tag, as the disulfide moiety facilitates adsorption to gold electrode surfaces, while the polyanionic phosphate groups facilitate interaction with polycationic electrochemical moietys, as described above. However, the presence of the terminal amine groups permits the detection tag to be further modified to include an osmium electrochemical moeity.

### Example 10

### Capture of Charged Tag on Unmodified Gold Electrode

PCR conditions are the same as described in Example 1 with the exception of the reporter probe, which has the following sequence, wherein DTPA is a disulfide-containing phosphate linker of the type shown in Scheme 20, and the 3 DTPA units in the probe promote chemisorption to the electrode surface without a hybridization event:

(DTPA)(DTPA)(DTPA) CAC GAA TCA AAG CTC TCA ACG CCT
GCA AGT CCT
AAG ACG CCA (biotin)

Following PCR, the uncleaved probe and amplicons are then removed using the above protocol for the streptavidin coated Dynal magnetic beads.

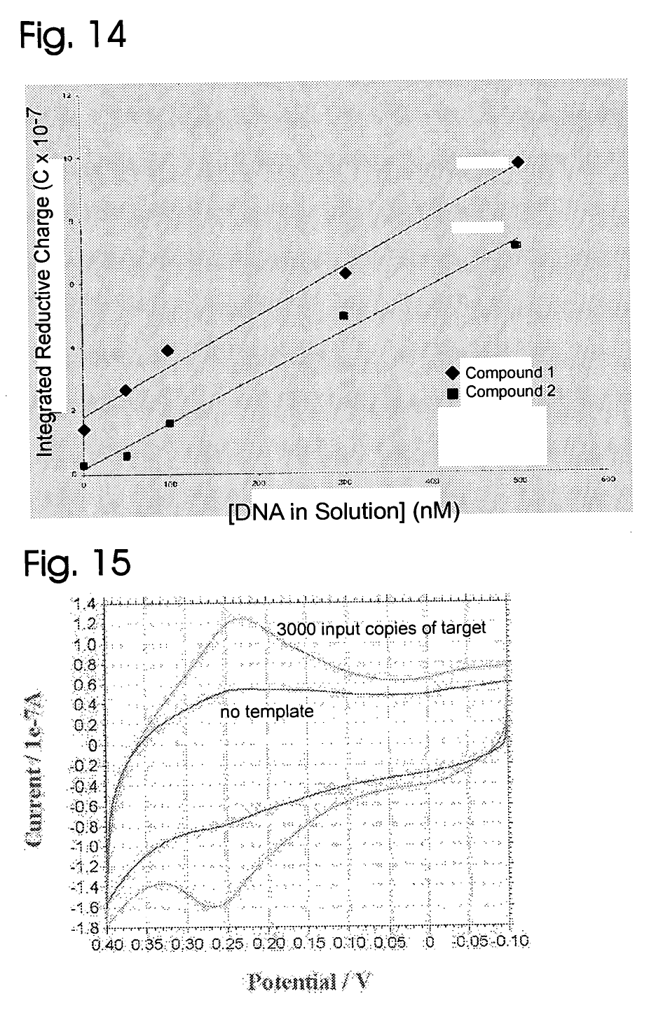

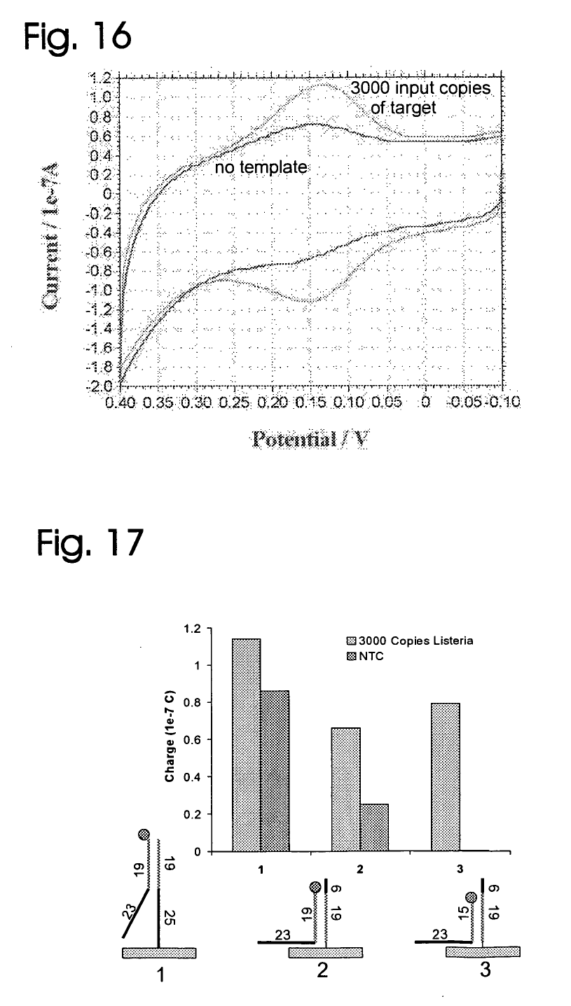

Electrodes were prepared and cleaned as previously described. After separation of the uncleaved probes and the amplicons using the streptavidin-coated Dynal Beads, the solutions are exposed to freshly cleaned gold electrodes for 20 minutes, followed by a 5 sec water rinse and exposure to a 1 mM aqueous solution of mecaptohexanol for 10 min. The electrodes are then exposed to aqueous solutions of the indicated cationic redox reporter molecules for 10 minutes and then rinsed for 20 sec in water. Cyclic voltammograms of solutions of 800 nM Probe and Compound 21 (shown in Scheme 10) were recorded vs. Ag/AgCl reference electrode and a platinum counter electrode, as shown in FIG. 15. Cyclic voltammograms of solutions of 800 nM Probe and Compound 7 were recorded vs. Ag/AgCl reference electrode and a platinum counter electrode, as shown in FIG. 16.

### Example 11

### Detection of Cleaved Tag in the Presence of Uncleaved Probe

This example illustrates embodiments in which a tag complement is immobilized on an electrode by thiol moieties (here provided by DTPA moieties) that exhibit specificity for binding to gold surfaces, such as a gold electrode, and a cleavable probe that contains (i) a polynucleotide sequence attached to the 5′ end of a target complementary segment and (ii) a detectable tag comprising an osmium-containing complex for electrochemical detection after capture of the cleaved tag by the immobilized tag complement.

The cleaved probe can be detected and/or measured in the presence of uncleaved probe by selection of an appropriate capture probe (a tag complement) such that the capture probe destabilizes capture of uncleaved (intact) probe by selectively binding the tag of the uncleaved probe close to the electrode surface. As a result, the capture probe hybridizes to the cleaved tag more stably than the uncleaved tag moiety bound to the probe.

A 50 μl reaction mix is prepared that contains 1× PCR buffer A (Applied Biosystems, P/N N808-0228), 6 mM MgCl₂, 200 μM of each dNTP, 200 nM of forward and reverse primers (see Example 1), 400 nM 5′-Os-labeled probe (see Scheme 21 below for Os complex labeling agent that was coupled to a 5′ amino group on each probe), 0.05 units of Gold AmpliTaq™ polymerase and 3,000 copies of Listeria monocytogenesis DNA.

Three different combinations of cleavable probes and immobilized tag complements were tested, as shown in the following combinations in which the upper sequence (underlined) represents the Os-labeled cleaved tag to be detected, and the lower sequence represents a capture probe that was attached to the electrode by 3 DTPA moieties at its 5′ end, and contained a tag complement for binding to the tag sequence:

Combination #1

| | |
|---|---|
| Tag | 5′-CACGAATCAAAGCTCTCAAX-3′ |
| 1: | |
| Cap | 3′ |
| 1: | GTGCTTAGTTTCGAGAGTTGTGTGAACTTAACGACCCCAAAAAAA5′ |

Combination #2

| | |
|---|---|
| Tag | 5′-CACGAATCAAAGCTCTCAAX-3′ |
| 1: | |
| Cap | 3′ AAAAAAGTGCTTAGTTTCGAGAGTT (C18) 5′ |
| 2: | |

Combination #3

| | |
|---|---|
| Tag | 5′-ATCAAAGCTCTCAAX-3′ |
| 2 | |
| Cap | 3′ AAAAAAGTGCTTAGTTTCGAGAGTT (C18) 5′ |
| 2: | |

Wherein X = CGCCTGCAAGTCCTAAGACGCCA-3′ (target-
specific segment) and C18 = (OCH₂CH₂)₆(DTPA)₃

Thermocycling was performed at 95° C. for 10 min., then (92° C. for 15 sec, 66° C. for 30sec.)×40 cycles. Then, the PCR mix is loaded into an electrochemical cell for electrochemical measurements as described in Examples 1 and 2. The measurements were performed using the hybridization buffer from Example 2, at 31° C. (which is approximately 10 degrees below the melt temperature (Tm) of the 15-mer cleaved tag sequence in Combination #3 above as calculated using the Tm calculator program on IDT web site: www.idt.com: Results are shown in FIG. 16.

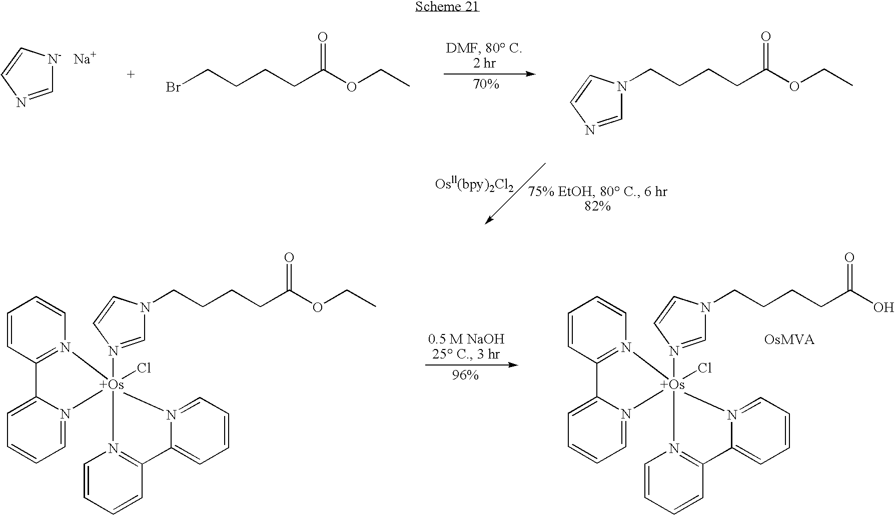

Although the present invention has been shown and described with reference to the foregoing operational principles and preferred embodiments, it will be apparent to those skilled in the art that various changes in form and detail can be made without departing from the spirit and scope of the invention. The present invention is intended to embrace all such alternatives, modifications and variances.

We claim:

1. A method of detecting a target polynucleotide sequence comprising:
contacting a probe with a sample comprising at least one target polynucleotide sequence under conditions effective for the probe to form a probe-target complex, wherein the probe comprises a target-complementary segment and a detectable tag,
cleaving the detectable tag from the probe,
associating the released tag with a tag complement that is coupled to an electrode to form an immobilized tag:tag complement complex at the electrode;
detecting an electrochemical signal that is related to the presence of the tag:tag complement complex; and
correlating the detected signal with the presence of the target polynucleotide sequence in the sample.

2. The method of claim 1, further comprising correlating the detected signal with an amount of the target polynucleotide sequence in the sample.

3. The method of claim 1, wherein cleaving the detectable tag includes cleaving the detectable tag enzymatically.

4. The method of claim 3, wherein said cleaving comprises cleaving the hybridized probe with a nuclease enzyme.

5. The method of claim 4, wherein said probe is cleaved during primer extension, the method further comprising amplifying the target sequence by a polymerase chain reaction.

6. The method of one of claims 3 to 5, wherein said cleavage is mediated by a 5′ nuclease activity of a DNA polymerase enzyme.

7. The method of one of claims 3 to 5, wherein the probe comprises an RNA segment, and said cleaving includes combining the probe-target complex with an enzyme having RNase H activity.

8. The method of any one of the preceding claims, further comprising:
hybridizing a second probe with the target polynucleotide; and
cleaving a second detectable tag from the hybridized probe.

9. The method of any one of the preceding claims, wherein the released detectable tag comprises a non-target-complementary polynucleotide sequence, and the tag complement comprises a polynucleotide sequence that is complementary to said non-target-complementary polynucleotide sequence.

10. The method of any one of the preceding claims, wherein the released detectable tag comprises an L-DNA polynucleotide sequence, and the tag complement comprises an L-DNA polynucleotide sequence that is complementary to the L-DNA polynucleotide sequence in the released detectable tag.

11. The method of any one of the preceding claims, wherein the electrochemical signal is generated using an electrochemical mediator that is associated with a double-stranded region in said tag:tag complement complex.

12. The method of any one of the preceding claims, wherein the released detectable tag comprises a polynucleotide moiety that is capable of forming a complex with said tag complement.

13. The method of any one of claims 1-11, wherein the released detectable tag comprises a non-polynucleotide moiety that is capable of forming a complex with said tag complement.

14. The method of claim 13, wherein the non-polynucleotide moiety is negatively charged and the tag complement is positively charged.

15. The method of claim 13, wherein the tag complement comprises a polycationic moiety.

16. The method of claim 15, wherein the polycationic moiety comprises a plurality of positively charged nitrogen-containing heterocyclic moieties.

17. The method of claim 13, wherein the non-polynucleotide moiety comprises one or more sulfhydryl groups, and the tag complement comprises gold.

18. The method of claim 13, wherein the non-polynucleotide moiety comprises a polyanionic moiety, and said electrochemical signal is generated using a polycationic electrochemical mediator that is electrostatically bound to said polyanionic moiety.

19. The method of any one of the proceding claims, wherein said cleaving is performed in the absence of the tag complement.

20. The method of any one of the preceding claims, wherein said contacting comprises contacting the sample with a plurality of probes that are each complementary for a distinct target polynucleotide sequence, and said detecting comprises detecting at least two different target sequences.

21. The method of any one of the preceding claims, wherein the concentration of said probe is equal to or less than 800 nM.

22. A microfluidic device for detecting a target polynucleotide sequence as claimed in any of claims 1-21, comprising
a substrate having a plurality of microfluidic chambers and channels fabricated therein;
a cover adhering to the substrate surface;
an inlet configured to receive a sample containing at least one target polynucleotide sequence;
one or more chambers configured for contacting a probe with the sample, wherein the probe comprises a target-complementary segment and a detectable tag,
one or more chambers configured for subjecting the sample to temperature control, cleaving the detectable tag from the probe, and associating the released tag with a tag complement that is coupled to an electrode to form an immobilized tag:tag complement complex; and
instrumentation configured for detecting an electrochemical signal that is related to the presence of the tag:tag complement complex; and
correlating the detected signal with the presence of the target polynucleotide sequence in the sample.
